(19) United States
(12) Patent Application Publication
(10) Pub. No.: US 2004/0219532 A1
(43) Pub. Date: Nov. 4, 2004

(54) Internal references measurements

(76) Inventors: Jeffrey R. Sampson, San Francisco, CA (US); Joel Myerson, Berkeley, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/426,490

(22) Filed: Apr. 30, 2003

(51) Int. Cl. C12Q 1/68

(52) U.S. Cl. 435/6

(57) ABSTRACT

The present invention provides an improved method of detecting differential expression of a gene of interest using modified nucleotides that reduce the levels of secondary structure in a nucleic acid molecule. In certain embodiments of the invention, multiple genes of interest are provided on the surface of a solid support, such as in the form of a microarray. The presence of carefully chosen unstructured nucleic acid bases (UNAs) in the samples being assayed and in the probes on the surface of the solid support provides an internal referenced measurement that is suitable for detecting the differential expression of a gene of interest in the samples. Also provided are arrays of pairs UNA probes that are capable of detecting differential expression of a particular gene of interest in two samples of nucleic acid.

### BACKGROUND OF THE INVENTION

Microarrays of binding agents, such as oligonucleotides, are important tools in the biotechnology industry and related fields. Typically a plurality of binding agents is deposited onto a solid support surface in the form of an array o-Or pattern. These microarrays find a variety of applications, including drug screening, oligonucleotide sequencing, and the like. Another important use of the microarrays is in the analysis of differential gene expression, where the expression of genes in different cells, normally a cell of interest and a control cell, is compared and any discrepancies in expression are identified. In such assays, the presence of discrepancies indicates a difference in the classes of genes expressed in the cells being compared. Such information is useful for the identification of the types of genes expressed in a particular cell or tissue type.

Perhaps one of the most significant alterations that can occur in a cell is a change in its pattern of gene transcription that exerts control of cellular protein levels and activities. Developing methods to detect alterations in transcription in biological samples is key to increasing our knowledge about the causes of diseases, the processes of cellular development and differentiation, and other physiological and cellular events. Such methods will also assist in the development of tools to detect, treat, alter, and monitor these conditions. The detection of changes in mRNA levels in one or thousands of genes expressed by a single cell is an important goal for many research programs.

A variety of mechanisms for detecting a specific mRNA on a microarray of mRNAs are available. These methods can be categorized into two general classes. One class requires a dual label approach in which the hybridization by a mixture of individually labeled samples is detected, and ratios of simultaneously detected hybridized labels are determined in a single operation in order to determine the expression level of a single gene. This approach requires that two distinguishable labels be used. The second class of method requires two separate hybridizations on two separate arrays, followed by subsequent signal ratio determination. This class of methods includes those in which target labels on the two samples are not distinguishable. Also included in the second class are methods in which the samples are not directly labeled.

With respect to the first class, two different mRNA samples, which are to be compared, are each labeled in separate reactions in vitro with one of two dyes (e.g., a cyanine dye, such as Cy3, and Cy5). The labeled samples are then mixed together and hybridized to an array of oligonucleotide sequences representative of one or more genes of interest. The relative mRNA expression levels of the two samples can then be directly compared by determining for each gene on the array the ratio of the dye fluorescence intensity. This “two color” method is advantageous in that it allows for direct comparisons between two related samples. However, one major drawback is that a dye, or some type of detector moiety, must be directly incorporated into the target molecules. This prohibits the use of “two color” or “dual label” assays with methods that cannot separate the two samples from one another. For example, using electrochemical detection, two target molecules on a single gene cannot be individually detected. Similarly, use of the two-color detection system is not feasible using elipsometric, gravimetric, or any other method in which the amount of one sample cannot be determined in the presence of another.

The use of two color detection systems can also be problematic in sample preparation, hybridization, or detection. For example, during dye incorporation, different proportions of the dyes can be incorporated into the two samples. During hybridization, different dyes can cause different efficiencies of hybridization. During detection, quenching artifacts, or phenomena that result in apparent quenching, can be different for the two different samples. Such inconsistencies can lead to inaccurate ratio determinations ultimately causing variable and incorrect results.

With respect to the second class of methods for detecting a specific mRNA on a microarray, some methods are particularly suited for direct detection of unlabeled samples. Such methods use pre-labeled array probes in which the hybridization event is detected either by some type of sample-dependent fluorescence quenching or analogous change in the electrical property of the array probe. Another common method is direct detection of hybridization through the use of dyes that are specific for dsDNA. Other methods for direct detection include sandwich assays, particularly those in which a secondary probe is reacted with the sample already bound to the surface of the array. This secondary probe may be coupled to any of a variety of different detection moieties, including those that utilize fluorescence, nanoparticle formation, chemiluminescence, and/or enzymatic amplification. The advantage of such detection methods is that they are more sensitive than traditional antibody labeling methods, which are limited by the incorporation efficiency of the label into the sample. However, this attribute is offset by the fact that the above methods, as well as other non-dual label methods, require two separate array assays to be performed, and thus extraordinary care to be taken in order to obtain valid ratio information.

There remains a need for improved methods and reagents for accurately and reliably detecting a binding event on a microarray that can be measured in a single assay.

### SUMMARY OF THE INVENTION

The present invention provides systems for detecting a binding event between probes and target samples. As a first step, unstructured nucleic acids (UNA) targets are derived from at least a first and second sample and contacted with a first and second UNA probe that is complementary to a gene of interest that is likely to be expressed in both the first and second samples. In preferred embodiments, the UNA target derived from the first and second samples differ in their UNA base content and are likely to comprise the same gene of interest. According to the invention, the sequence and composition of the first UNA probe is selected to specifically hybridize to the UNA target derived from the first sample for the gene of interest and not to the UNA target derived from the second sample for the same gene of interest. Similarly, the sequence and composition of the second UNA probe is selected to specifically hybridize to UNA targets derived from the second sample for the gene of interest in and not to the UNA target derived from the first sample for the same gene of interest. As a second step, the relative extent of hybridization between the UNA target derived from the first and second samples and their respective first and second UNA probes is detected. The specificity of hybridization is due to the complementarity of UNA bases between the first UNA probe and the UNA target derived from the first sample, and the second UNA probe and UNA targets derived from the second sample.

In certain preferred embodiments, a plurality of probe pairs which correspond to a plurality of genes of interest are provided. In other preferred embodiments, sets of probes for a plurality of genes of interest are provided. As used herein, a pair of probes includes two probes that recognize a UNA target corresponding to the same gene of interest derived from two different samples. A set of probes includes more than two probes that recognize a target UNA corresponding to the same gene of interest derived from two or more different samples. Preferably, the pairs or sets of probes are contacted with the target samples in the same vessel. In certain embodiments, the step of contacting the first and second samples with the first and second probes occurs simultaneously.

Exemplary UNA targets of the present invention are derived from samples containing, for example, DNA or RNA molecules (e.g., mRNA molecules). Exemplary UNA probes of the present invention include UNAs comprising, for example, DNA, RNA or PNA. In some embodiments, the UNA probes are associated with the surface of a solid support. The UNA probes may be labeled or unlabeled. One particularly preferred class of probe labeling and related detection method is that in which the labeled probe enables target-binding dependent detection. This would include, for example, electronic detection methods which employ electrochemical or redox-active probes and fluorescence detection methods which employ intercalating dyes.

In related embodiments, the present invention provides systems for detecting differentially expressed genes by; a) providing UNA targets derived from at least a first and second sample, wherein the UNA targets derived from the first and second samples comprise different UNA nucleotides for the same gene of interest; b) providing a pair or a set of UNA probes for at least one gene of interest including at least a first UNA probe and a second UNA probe, wherein the sequence of the first UNA probe is selected to specifically hybridize the UNA target corresponding to the gene of interest derived from the first sample and not to the UNA target corresponding to the same gene of interest derived from the second sample, and the sequence of the second UNA probe is selected to specifically hybridize to the UNA target corresponding to the gene of interest derived from the second sample and not to the UNA target corresponding to the same gene of interest derived from the first sample; c) hybridizing the UNA targets derived from the first and second samples to an excess of copies of the first and second UNA probes; d) detecting the amounts of hybridization of the UNA target derived from the first sample to the first UNA probe and detecting amounts of hybridization of the UNA target derived from the second sample to the second UNA probe, a difference in the relative amount of hybridization of the UNA target derived from one sample compared to the other sample indicating that the gene of interest in one of the samples is differentially expressed.

The present invention further provides systems for producing arrays of UNA probes stably associated with the surface of a solid support. As described herein, the array contains pairs or sets of UNA probes having at least a first and a second probe, wherein the probes in the pairs or sets are composed of different UNA bases and are capable of detecting a UNA target corresponding to the same gene derived from different samples. In one preferred embodiment, the array is produced by a) selecting at least one gene that is likely to be present in different samples, and b) generating at least a pair of UNA probes that is complementary to some portion of the gene, wherein the first probe of the pair is capable of hybridizing to the UNA target corresponding to the gene derived from the first sample and not capable of hybridizing to the UNA target corresponding to the same gene derived from the second sample and the second probe of the pair is capable of hybridizing to the UNA target corresponding to the gene derived in the second sample and not to the UNA target corresponding to the same gene derived from the first sample.

In related embodiments, the present invention provides an array having a plurality of pairs or sets of UNA probes. In certain preferred embodiments, the plurality of UNA probes is stably associated with the surface of a solid support. In related embodiments, the present invention provides a kit for carrying out differential gene expression analysis that includes an array of UNA base-containing probe molecules on a planar support. Preferably, the probes are arranged on the planar support in a particular pattern. In the preferred embodiment, the array contains pairs of probes, wherein each member of a pair is capable of hybridizing to a UNA target corresponding to the same gene derived from different samples. Alternatively, the array contains sets of probes, wherein each member of a set is capable of detecting a UNA target corresponding to the same gene derived from three or more different samples. In the preferred embodiment, the array will include a plurality of probe pairs or sets wherein the different probe pairs or sets are complementary to UNA targets corresponding to different genes of interest within two or more samples, wherein each probe member of a particular pair or set specifically hybridizes to the UNA target derived from only one of two or more samples. All of the UNA targets derived from a given sample may or may not comprise the same UNA nucleotides. Arrays of the present invention include, e.g., multiwell plates or biochips. In certain preferred embodiments, the kit further includes one or more of the following: target sample generation reagents, reagents used in the binding step, target sample generation reagents, reagents used in the binding step, and signal producing system members.

The present invention also provides systems for detecting a binding event on a microarray that include at least one array probe attached to the array; targets derived from two or more samples, wherein the targets are UNAs that both compete for hybridization to the same arrayed probe; and two or more labeling probes each having a different UNA chemistry such that each labeling probe specifically hybridizes to the UNA target derived from a different sample, wherein each labeling probe is attached to a different detector moiety. In a preferred embodiment, the array contains a plurality of array probes, wherein each array probe is complementary to the UNA target corresponding to different genes of interest derived from two or more samples.

In related embodiments, the present invention provides methods and reagents for detecting a binding event on a microarray by providing at least one array probe on an array corresponding to a gene of interest; contacting the array probe with at least two targets derived from two or more samples, wherein the targets are UNAs that hybridize to the same array probe; contacting the UNA targets with labeling probes, wherein each labeling probe has a different UNA chemistry that directs specific hybridization to the UNA target derived from only one of the two or more samples, wherein the labeling probe is attached to a different dye that serves as a detectable marker for the target; and detecting the dyes that are hybridized to the targets, thereby detecting which target is bound to the array.

### DEFINITIONS

“Sequencing”: The term “sequencing” as used herein means determining the sequential order of nucleotides in a nucleic acid molecule. Sequencing as used herein includes in the scope of its definition, determining the nucleotide sequence of a nucleic acid in a de novo manner in which the sequence was previously unknown. Sequencing as used herein also includes in the scope of its definition, determining the nucleotide sequence of a nucleic acid of which the sequence was previously known. Sequencing nucleic acid molecules whose sequence was previously known may be used to identify a nucleic acid molecule, to confirm a nucleic acid sequence, or to search for polymorphisms and genetic mutations.

“Modified Nucleotide”: Nucleic acid bases may be defined for purposes of the present invention as nitrogenous bases derived from purine or pyrimidine. Modified bases (excluding A, T, G, C, and U) include for example, bases having a structure derived from purine or pyrimidine (i.e. base analogs). For example without limitation, a modified adenine may have a structure comprising a purine with a nitrogen atom covalently bonded to C6 of the purine ring as numbered by conventional nomenclature known in the art. In addition, it is recognized that modifications to the purine ring and/or the C6 nitrogen may also be included in a modified adenine. A modified thymine may have a structure comprising at least a pyrimidine, an oxygen atom covalently bonded to the C4 carbon, and a C5 methyl group. Again, it is recognized by those skilled in the art that modifications to the pyrimidine ring; the C4 oxygen and/or the C5 methyl group may also be included in a modified adenine. Derivatives of uracil may have a structure comprising at least a pyrimidine, an oxygen atom covalently bonded to the C4 carbon and no C5 methyl group. For example without limitation, a modified guanine may have a structure comprising at least a purine, and an oxygen atom covalently bonded to the C6 carbon. A modified cytosine has a structure comprising a pyrimidine and a nitrogen atom covalently bonded to the C4 carbon. Modifications to the purine ring and/or the C6 oxygen atom may also be included in modified guanine bases. Modifications to the pyrimidine ring and/or the C4 nitrogen atom may also be included in modified cytosine bases.

Analogs may also be derivatives of purines without restrictions to atoms covalently bonded to the C6 carbon. These analogs would be defined as purine derivatives. Analogs may also be derivatives of pyrimidines without restrictions to atoms covalently bonded to the C4 carbon. These analogs would be defined as pyrimidine derivatives. The present invention includes purine analogs having the capability of forming stable base pairs with pyrimidine analogs without limitation to analogs of A, T, G, C, and U as defined. The present invention also includes purine analogs not having the capability of forming stable base pairs with pyrimidine analogs without limitation to analogs of A, T, G, C, and U.

In addition to purines and pyrimidines, modified bases or analogs, as those terms are used herein, include any compound that can form a hydrogen bond with one or more naturally occurring bases or with another base analog. Any compound that forms at least two hydrogen bonds with T (or U) or with a derivative of T or U is considered to be an analog of A, or a modified A. Similarly, any compound that forms at least two hydrogen bonds with A or with a derivative of A is considered to be an analog of T (or U), or a modified T or U. Similarly, any compound that forms at least two hydrogen bonds with G or with a derivative of G is considered to be an analog of C or a modified C. Similarly, any compound that forms at least two hydrogen bonds with C or with a derivative of C is considered to be an analog of G or a modified G. It is recognized that under this scheme, some compounds will be considered for example to be both A analogs and G analogs.

“Hybridization”: Hybridization as used herein means the formation of hydrogen-bonded base pairs between two regions having substantially complementary sequences to form a duplex. Two complementary sequences do not have to be 100% complementary for duplex formation. Certain mismatches may be tolerated for hybridization to occur. Conditions that promote duplex formation or hinder duplex formation are well known to those of ordinary skill in the art. It is recognized that hybridization includes in its definition, transiently stable duplex which are stable long enough to be detected and/or to allow a biological process to occur (e.g. primer extension).

A stable base pair is defined as two bases that can interact through the formation of at least two hydrogen bonds. Alternatively or additionally, a stable base pair may be defined as two bases that interact through at least one, preferably two, hydrogen bonds that promote base stacking interactions and therefore, promotes duplex stability.

“Complementary”: Complementary bases are defined according to the Watson-Crick definition for base pairing. Adenine base is complementary to thymine base and forms a stable base pair. Guanine base is complementary to cytosine base and forms a stable base pair. The base-pairing scheme is depicted in FIGS. 1, 2 and 7. Complementation of modified base analogs is defined according to the parent nucleotide. Complementation of modified bases does not require the ability to form stable hydrogen bonded base pairs. In other words, two modified bases may be complementary but may not form a stable base pair. Complementation of base analogs which are not considered derivatives of A, T, G, C or U is defined according to an ability to form a stable base pair with a base or base analog. For example, a particular derivative of C (i.e. 2-thiocytosine) may not form a stable base pair with G, but is still considered complementary.

Complementary is also used to refer to nucleic acids containing UNA bases that can form stable base-pairs with only a defined subset of complementary bases. The complementary UNAs of the invention hybridize preferably to one another. For example, a UNA probe hybridizes to its complementary target nucleic acid sample.

A “target” is a nucleic acid that is being detected. According to the present invention, the target is usually a sample, within a sample or derived (e.g. copied, replicated or amplified) from a sample of nucleic acid. The target nucleic acid can be a DNA or RNA molecule of any species. The target nucleic acids of the invention are typically UNAs. The targets may be labeled with some type of detection moiety such as a dye, nano-particle or redox-active moiety, as well as moities, which can undergo signal amplification when used under appropriate conditions determined by one of ordinary skill in the art. The target is a molecule that hybridizes to a probe. In certain preferred embodiments, the target molecules of the invention hybridize to an array probe. In related embodiments, the target molecules hybridize to both an array probe and a labeling probe.

A “probe” is the nucleic acid used to detect the target. A probe molecule is capable of hybridizing specifically to a target molecule, under appropriate conditions determined by one of ordinary skill in the art. Probe molecules may be DNA, RNA, PNAs or mixtures thereof. Probe molecules of the invention may be UNA molecules that are complementary to the target UNA molecules as defined within the specifications.

“Array probe” is used herein to refer to a probe that is attached to a solid surface such as a microarray.

“Labeling probe” is used herein to refer to a probe that is labeled with some type of detection moiety such as a dye, nano-particle or redox-active moiety, and includes moieties, which can undergo signal amplification when used under appropriate conditions determined by one of ordinary skill in the art. The labeling probes of the invention preferably hybridize to a target molecule, which target molecule may be hybridized to an array probe.

“Naturally occurring bases”: Naturally occurring bases are defined for the purposes of the present invention as adenine (A), thymine (T), guanine (G), cytosine (C), and uracil (U). The structures of A, T, G and C are shown in FIGS. 1, 2 and 7. For RNA, uracil (U) replaces thymine. Uracil (structure not shown) lacks the 5-methyl group of T. It is recognized that certain modifications of these bases occur in nature. However, modifications of A, T, G, C, and U that occur in nature are also considered to be non-naturally occurring. For example, 2-aminoadenosine is found in nature, but is not a “naturally occurring” base as that term is used herein. Other non-limiting examples of modified bases that occur in nature but are considered to be non-naturally occurring are 5-methylcytosine, 3-methyladenine, 0(6)-methylguanine, and 8-oxoguanine.

A “binding event” is the occurrence of an interaction between a binding agent, e.g., a probe, and a target, e.g., an mRNA. According to the invention, the target may include a target compound such as a target UNA sample (e.g., a sample of UNA mRNAs) and the binding agent may include a collection of UNA probes arrayed onto the surface of a solid support. In this example, the binding event may occur between particular molecules in the target sample of mRNAs, and particular probes in the array. Other binding events may occur between a target DNA and an array probe, a target enzyme and its substrate, an antibody and its target peptide, and the like.

### DESCRIPTION OF THE DRAWINGS

FIG. 1 is a schematic depicting base pairing between naturally occurring and UNA bases.

FIG. 2 is a schematic depicting base pairing between naturally occurring and UNA bases.

FIG. 3 is a schematic depicting the use of UNA targets and mutually exclusive hybridizing UNA probes for performing an internal reference measurement.

FIG. 4 is a schematic depicting the use of UNA targets and mutually exclusive hybridizing UNA probes for performing an internal reference measurement on an array surface.

FIG. 5 is a schematic depicting the use of UNA targets and UNA probes having defined nucleotide compositions for performing a gene expression analysis.

FIG. 6 is a diagram illustrating a “two-color sandwich assay” where the hybridization of two different targets, having different UNA chemistries, to a probe on an array and detection of the hybridization using two different labeling probes that are labeled with two different dyes (1 and 2).

FIG. 7 is a schematic depicting base pairing between naturally occurring and UNA bases

### DESCRIPTION OF CERTAIN PREFERRED EMBODIMENTS

The present invention provides methods and reagents for detecting a binding event in a single assay. Generally, the present invention provides methods and reagents for detecting a binding event between a binding agent and a target using internal referenced measurements. In preferred embodiments, the present invention is directed to methods and reagents for detecting molecular alterations in biological samples. The present invention is particularly useful for measuring changes in patterns of gene transcription using internally referenced samples of mRNA that hybridize specifically to internally referenced probes of one or more different genes. Specifically, the invention employs complementary internally referenced unstructured nucleic acids (UNAs) as targets and probes to identify alterations in gene transcription.

UNAs are a class of nucleic acids in which the nucleotide bases are modified such that they can form stable base pairs with only a defined subset of complementary bases. The base pairing concepts of UNAs are schematically depicted by the following formulas where A′≠T′ and G′≠C′ represent disallowed base-pairing schemes, with the symbol≠ representing the inability to form a base pair. [A*, T*, G*, and C*] represent a second group of bases capable of forming base pairs with A′, T′, G′ and C′ according to the general Watson-Crick base pair scheme of A=T and G=C, where=represents the ability to form a base pair. The same base pairing rules apply for RNA where U replaces T. (The horizontal base pairing symbols are not meant to represent the number of hydrogen bonds present in the base pair, but are meant only to indicate a stable base pair or lack of a stable base pair.)

(A′≠T′; G′≠C′) (1)

(A′=T*; T′=A*; G′=C*; C′=G*) (2)

Formula 1 indicates that base pair analogs A′/T′ and G′/C′ are unable to form a stable base pair. However, as indicated in Formula 2, the bases of nucleotides A′T′G′ and C′ are capable of forming stable base pairs with a second group of nucleotide bases (A*T*G*C*). In addition, the second set of nucleotide bases (A*T*G*C*) may or may not retain their ability to form stable base pairs with their respective complement.

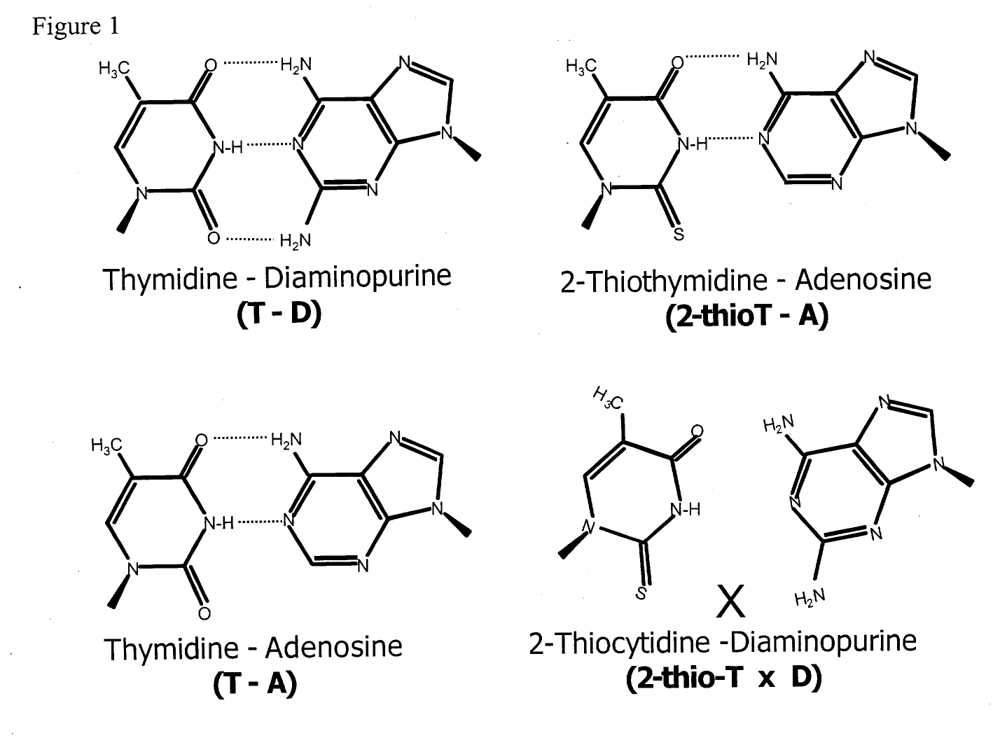

Examples of known UNA base pairing are described in greater detail below. It is known that adenine (A) can form a stable base pair with 2-thiothymine (2-thioT) (A=2-thioT) and 2,6-diaminopurine (D) can form a stable base pair with thymine (T) (D=T). However, 2,6-diaminopurine (D) and 2-thiothymine (2-thioT) cannot form a stable base pair. (D≠2-thioT). (see FIG. 1).

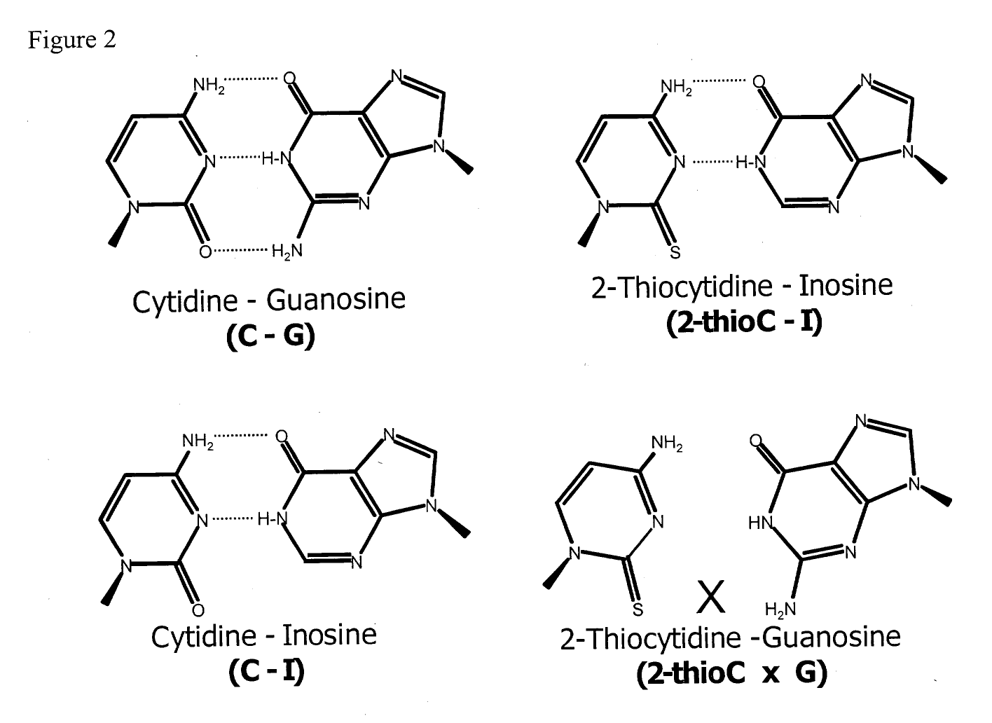

Likewise, both guanine (G) and inosine (I) can form stable base pairs with cytosine (C) (G=C and I=C). However, guanine (G) cannot form stable base pairs with 2-thiocytidine (2-thioC) (G≠2-thioC). (see FIG. 2).

Target UNAs of the present invention are generated by enzymatically incorporating modified nucleotide triphosphates that have a reduced ability to form base pairs with complementary modified and unmodified nucleotides. Preferably, the target UNAs are generated from a template containing complementary unmodified nucleotides. However, it is within the scope of the present invention for the template to contain other modified nucleotide complements that do form base pairs with the target UNA in order for the template to be used by enzymes for nucleotide incorporation into target UNAs.

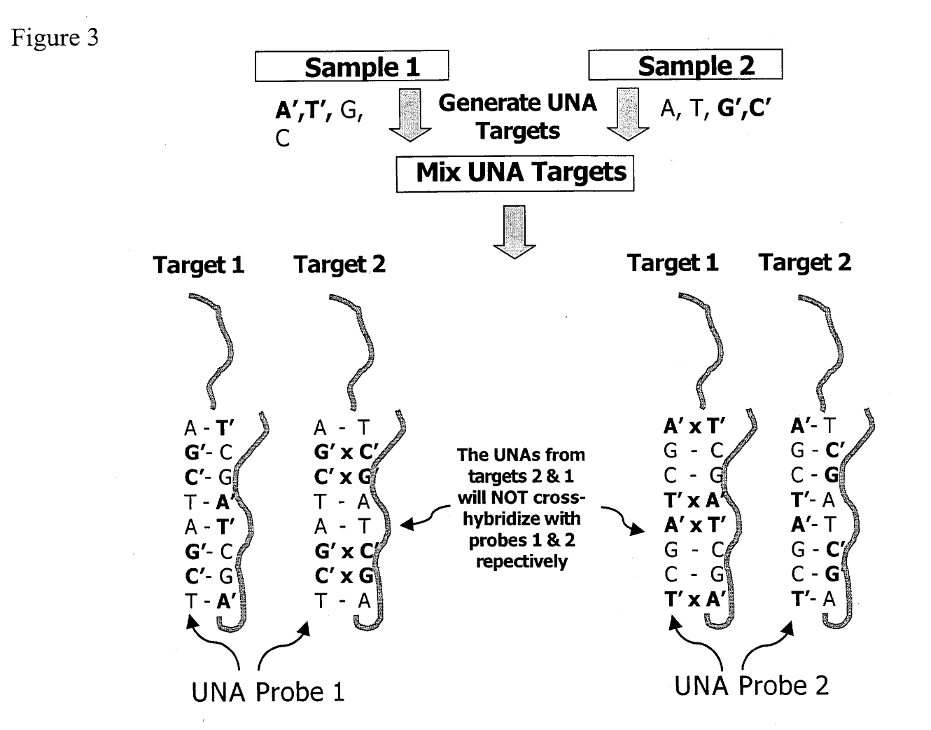

In one aspect, the present invention utilizes UNA targets and UNA probes to directly compare one or more nucleic acids derived from two or more samples in a single assay mixture. Nucleic acids of the invention include DNA and RNA (e.g., mRNA) and any modifications or derivatives thereof as recognized by the skilled artisan. In one embodiment, the present invention utilizes UNA targets derived from mRNAs within two or more samples and probes that are UNA oligonucleotides. According to the invention, one or more probes that are complementary to a particular gene of interest is generated for each sample wherein each probe is capable of hybridizing to the UNA target derived from only one of the two or more samples in the assay. The sample-specific hybridization for each target-probe interaction is generated by the UNA nucleotide content of both the probe and the UNA derived from the mRNA within the samples. More particularly, the specificity of hybridization is due to the reduced ability of a particular UNA probe to form stable base pairs with its complementary UNA target in all but one of the samples in the assay (FIG. 3).

More particularly, a UNA probe is generated for a particular UNA target that is derived from the mRNA from one of two or more samples such that hybridization between the probe and the UNA derived from a given sample is exclusive. A gene expression ratio for the particular mRNA species can then be determined by measuring the relative amount of hybridization of each UNA that was derived from the two or more samples to its respective complementary UNA probe. In this way, the level of transcription of the particular gene of interest from multiple samples can be simultaneously quantitated and compared.

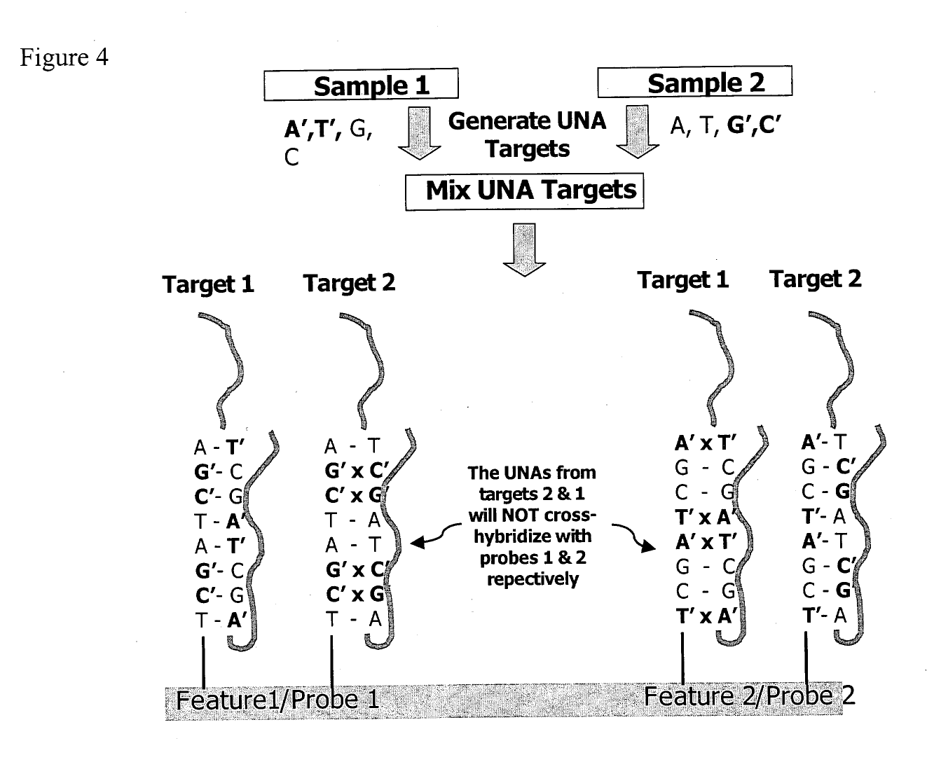

In preferred embodiments, the UNA probes are associated with the surface of a solid support such as an array. The array contains pairs of UNA probes having at least a first and a second probe, wherein the probes in the pairs are composed of different UNA nucleotides and are capable of detecting a UNA target corresponding to the same gene derived from different samples (FIG. 4).

In certain preferred embodiments, a single array contains sets of probes where a set of probes corresponds to a member of a multiple set of genes, so that the expression profile of the multiple set of genes present within two or more samples may be determined in one measurement. More particularly, for each UNA target species that is derived from a particular set of mRNAs that is present within one of two or more samples, a probe is generated such that hybridization between the probe and a given UNA target species derived from a particular sample is exclusive. The gene expression ratios for all the mRNA species correspond to the UNAs in the various two or more samples can then be determined simultaneously by measuring the relative amount of hybridization of each UNA target to its respective complementary UNA probe. In this way, the level of transcription of multiple genes of interest from multiple samples can be simultaneously quantitated and compared. In preferred embodiments, the present invention provides methods of detecting differentially expressed genes in two or more target samples. According to the present invention, the UNA targets comprise a particular combination of UNA nucleotides that is unique to the targets in that particular sample, and is not present in the other UNA targets derived from the other samples. All of the targets derived from a particular sample may or may not comprise the same combination of UNA nucleotides. The methods include the steps of contacting the UNA targets derived from two or more samples with a collection of probes that are UNA probes. As described herein, the UNA nucleotide content of each UNA probe is selected so that each UNA probe specifically hybridizes the corresponding UNA target of interest derived from one of the samples and does not hybridize to the UNA target derived from the other samples. Identification of a hybridization event between the UNA targets and a probe is detected using any of a variety of methods, as described herein.

As but one example, according to the present invention a first sample and a second sample of target UNAs is contacted with a first and second set of UNA probes that hybridize to their respective complementary UNA targets, preferably where more than one gene of interest, most preferably a plurality of genes of interest are represented by UNAs in the first and second samples. The UNA nucleotide content of both the first and second probes and the first and second UNA target samples is selected such that the first UNA probe specifically hybridizes to UNA target corresponding to the gene of interest derived from the first sample and does not hybridize to the UNA corresponding to the same gene of interest derived from the second sample. Likewise, the second UNA probe is designed to specifically hybridize to the UNA corresponding to the gene of interest derived from the second sample and does not hybridize to the UNA corresponding to the same gene of interest derived from the first sample. A hybridization event between the UNA targets derived from the first and second sample and their respective first and second UNA probes may then be detected.

Thus, the present invention utilizes UNA chemistry to generate an internal referenced mRNA measurement. For example, mRNA from a first target sample is copied into cDNA comprising defined UNA nucleotides using a DNA polymerase or reverse transcriptase in the presence of the UNA 2′-deoxynucleotide triphosphates, dDTP, dGTP, d-2-thioTTP and dCTP (see EP 1072679). The mRNA from a second target sample is similarly copied into a cDNA comprising a second combination of UNA nucleotides using a DNA polymerase or reverse transcriptase in the presence of the UNA nucleotide triphosphates dATP, dGTP, dTTP and d-2-thioCTP. A similar UNA-based internal referenced measurement method can also be accomplished using fewer UNA nucleotides. For example, the first sample can be copied into a CDNA in the presence dATP, dGTP, d-2-thioTTP and dCTP, while the second sample can be copied into a cDNA using dATP, dGTP, dTTP and d2-thioCTP. In this particular case however, the first target molecules will not have reduced intramolecular structures but will retain their ability to have the desired selective hybridization properties as specified in the present invention. These same schemes hold true for the UNAs comprised of ribonucleotides as well.

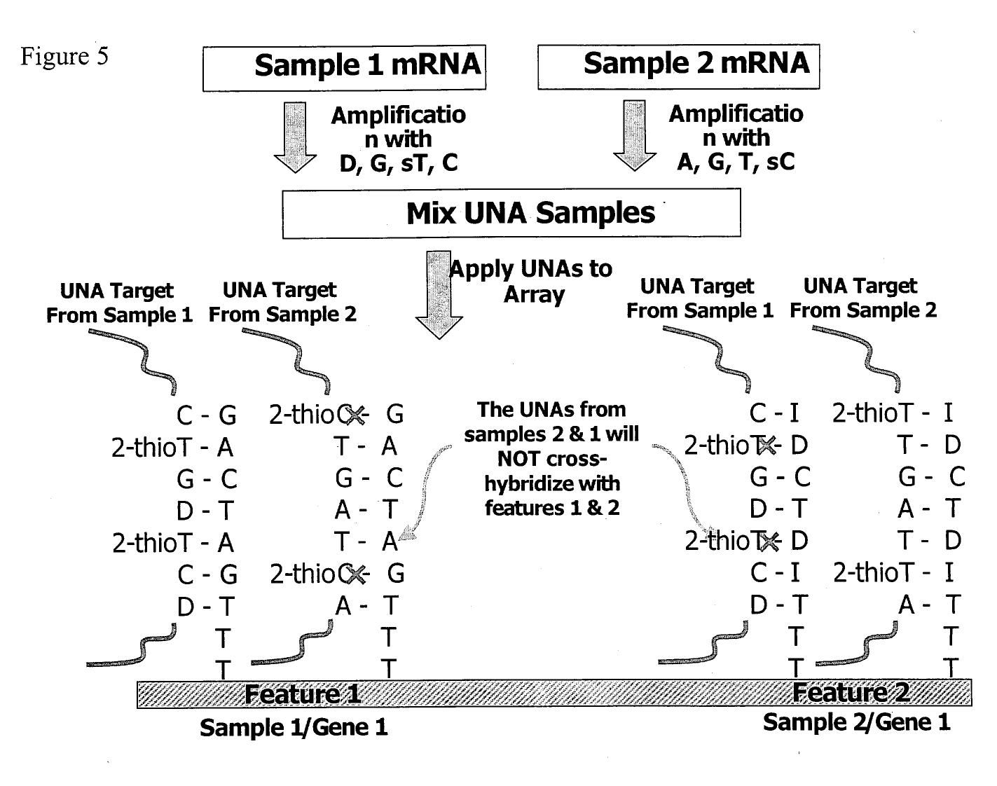

Once the first and second target samples are amplified, they are mixed together and hybridized to an array containing pairs of UNA probes in which each pair is designated to a particular gene of interest. Most importantly, the individual probes within each pair of will differ from one another in their UNA content such that the amplified UNA target from the first sample hybridizes to only one of the probes, while the amplified UNA target from the second sample hybridizes to only the other probe (FIG. 5).

The invention allows two or more related, yet separately amplified target samples to be directly compared on one array. The advantage of the present invention over the existing two color labeling systems is that the present invention negates the need to incorporate labels into the target nucleic acid samples being analyzed. An additional advantage of the present invention over the existing two color labeling systems is that the present method allows for a simultaneous comparative analysis method for two or more samples even when the detection method (e.g. electrical detection or surface plasmon resonance) is unable to distinguish between two types of chemical entities (e.g. target samples).

As described herein, for the present inventive methods to perform adequately, the probe molecules must be carefully designed with the appropriate nucleotide composition such that the targets derived from the two or more samples can be distinguished by their ability to hybridize with the appropriate UNA probe. For example, hybridization between the first probe and the UNA target derived from the first sample is favored (perfect duplex formation), whereas hybridization between the first probe and the UNA target derived from second sample is disfavored (UNA-directed mismatch duplex formation). As shown in FIGS. 3-6, based on the UNA content of the targets derived from the first and second samples and the content of first and second probes, the amplified UNAs derived from the first sample will not cross hybridize to the second probe and vice versa.

The nature of UNA-DNA or UNA-RNA interactions is such that they may be stronger than naturally occurring DNA-DNA interactions or naturally occurring RNA-RNA interactions. The desired hybridizations will be enhanced by this phenomenon, while incorrect hybridizations are not only lacking these interactions, but have the destabilizing UNA-UNA interactions.

In a related embodiment, the present invention provides methods of detecting differentially expressed genes by a) providing target UNAs derived from a first sample and target UNAs derived from a second sample; b) providing a pair of UNA probes corresponding to least one gene of interest present in both samples that include a first UNA probe and a second UNA probe, where the first UNA probe is capable of specifically hybridizing to the UNA target corresponding to the gene of interest in a first sample and not the UNA target corresponding to the same gene of interest in a second sample and the second UNA probe is capable of specifically hybridizing to the UNA target corresponding to the gene of interest in the second sample and not UNA target corresponding to the gene of interest the first sample; c) hybridizing the UNA targets derived from the first sample to an excess of copies of the first UNA probe and hybridizing the UNA targets derived from the second sample to an excess of copies of the second UNA probe; and detecting the amounts of hybridization of the UNA targets from the first sample to the first UNA probe and detecting the amounts of hybridization of the UNA targets from second sample to the second UNA probe. According to the invention, differential gene expression between the two samples for a particular gene of interest is detected by observing a difference in the amount of hybridization between the probes and their respective complementary UNA targets from one sample compared to that of the other sample.

According to certain preferred embodiments where more than two samples are assessed for the presence of a particular gene, multiple UNA probes are generated wherein each probe is specific for the UNA target in the particular sample. Those skilled in the art will appreciate that a large number of sample-probe pairs may be generated for a particular gene as long as each probe specifically hybridizes to the UNA target derived from a particular sample based on the unique UNA content of the UNA target and the probe.

Detection of a hybridization event between a UNA probe and a UNA target sample may be accomplished by any of a variety of methods. The present invention provides an “internal referenced measurement” to obtain a gene expression profile that is not subject to the detection limitations of a standard two-color assay. The methods of the present invention are particularly suitable for use with detection systems that previously required the use of two separate arrays for gene expression profile determination.

In one embodiment, both the UNA target and probe are unlabeled. Detection can be performed using methods that directly detect binding due to changes in properties such as refractive index or mass. Examples of such methods include surface plasmon resonance (SPR), ellipsometry, surface acoustic wave detection, and nanomechanical cantilever based methods (e.g. McKendry et.al. Proc Nat Acad Sci, USA (2002) 99: 9783-9788).

In another embodiment, the UNA target is unlabeled and the probe is labeled with a detectable moiety. One particularly preferred type of label is a label whose detection is dependent on a hybridization event between the target and the probe. The step of detecting may be accomplished by labeling the probe and detecting changes in the properties of the label. For example, the present invention may utilize pre-labeled array probes in which a hybridization event is detected by either target-dependent fluorescence or a change in the electrical property of the probe (see for example; Ihara et al., Nucleic Acids Res. (1996), 24, 4273-4280).

In another embodiment, the probe is initially unlabelled, but becomes labeled as a consequence of the target hybridization. For example, a primer extension reaction can be performed on the free 3′-end of an array probe, using mixture of fluorescent ddNTPs. The extension reaction serves to label the probe during or after the desired hybridization event (J. M. Shumaker et al., Hum. Mutation (1996) 7:346-354)

In another embodiment, the target sample is labeled and the probe is unlabeled. For example, both UNA target samples could be labeled with identical detectable moieties, such as a fluorophore or biotin. This embodiment is an example of a one-color internal referenced measurement.

In yet another embodiment, both the target sample and the probe contain components of the label.

Other detection methods that may be utilized in the present invention include the use of dyes that are specific for double stranded nucleic acids, for example, fluorescence methods which employ double-strand specific intercalating dyes (C. T. Wittwer et al., BioTechniques (1997) 22:130 & M. Jobs et al, Analytical Chem. (2002) 74:199-202. Alternatively, immunological assays, which utilize a secondary antibody specific for the sample that is to be detected (e.g., double stranded nucleic acid, e.g., target mRNA already bound to the surface of the array) can be used. Those skilled in the art will appreciate that this secondary probe can be coupled to many different detection moieties, including fluorescence, nanoparticle formation, chemiluminescence, and enzymatic amplification.

It will also be appreciated that the present type of UNA-based internal referenced measurement can be used with formats other than arrays, such as beads and particles. Furthermore, the present method can be preformed in conjunction with a variety of detection schemes that differentiate between double and single stranded nucleic acid, e.g., DNA. These include biotin/streptavidin and nanogold labeling, discussed in further detail below.

The present invention further provides a method of producing an array of UNA probes stably associated with the surface of a solid support such as an array. In certain preferred embodiments, the array includes pairs of UNA probes that are capable of hybridizing to the UNA targets corresponding to a particular gene of interest. The members of the pair of UNA probes differ in UNA base content such that each probe is complementary to a particular UNA target derived from a particular sample. In other preferred embodiments, the array contains a plurality of UNA probes. For example, the array may contain sets of UNA probes containing multiple members, where each member is complementary to a different UNA target derived from the same or different samples.

As but one example, an array of a plurality of probes includes UNA probes arranged in pairs wherein the members of a pair are capable of hybridizing to a UNA target corresponding to the same gene. The UNA probes, labeled such that the first member of the pair of UNA probes can be differentiated from the second member of the pair, are stably associated with the surface of a solid support. Preferably the first member of the pair is capable of hybridizing to a UNA target derived from the first sample and the second member of the pair is capable of hybridizing to a UNA target derived from the second sample. According to the present example, the UNA probes are arranged on the solid support according to pairs. For example, the pairs may be arranged such that the location of each member of the pair on the solid support is known.

The method of producing an array of UNA probes stably associated with the surface of a solid support includes the steps of 1) selecting a plurality of genes to be interrogated, and 2) generating at least a pair of UNA probes for the UNA targets corresponding to each gene of interest, wherein each pair of UNA probes is directed at different UNA target species in the samples. Where a pair of UNA probes for a particular gene is employed, the probes contain UNA nucleotides so that the first member of the pair is capable of hybridizing only to the UNA target corresponding to the gene of interest derived from the first sample and the second member of the pair is capable of hybridizing to the analogous UNA target derived from the second sample. In certain embodiments the probes are labeled to enable detection. Particularly preferred labels are those that allow target binding-dependent detection.

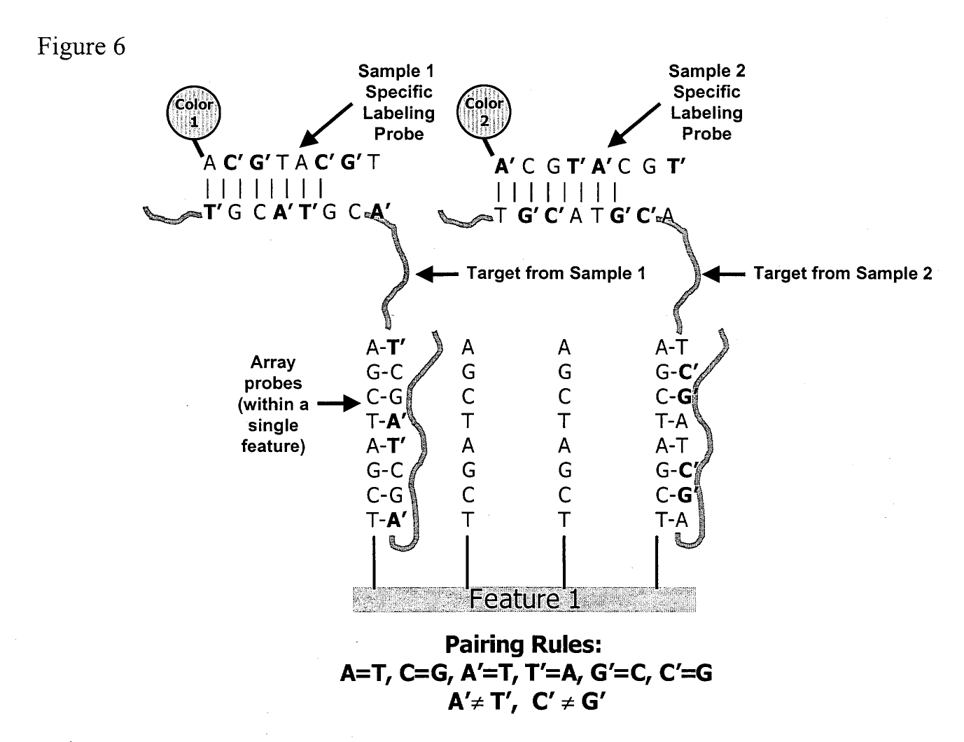

In alternative embodiments, the present invention provides methods and reagents for detecting hybridization events on a microarray that employ a two-color “sandwich assay”. In this particular embodiment, the composition of the array probes are such that they can hybridize, with approximately equal efficiencies, UNA targets derived from both the first and second sample. The detection, discrimination and hence determination of the relative ratios of the hybridized UNA targets from the two sample sources is carried out by the hybridization of secondary labeling probes, referred to herein as “labeling probes”. The labeling probes possess a unique detection moiety (e.g. fluorescent dye) and a have unique UNA chemistries that directs specific hybridization to the target(s) from only one of the two samples in the hybridization mixture (see FIG. 6). The labeling probes may be complementary to the same or different regions of the targets in the hybridization mixture. Due to the specificity of the labeling probe for the UNA target from their respective sample, there is no need for a different array probe for each of the UNA targets, thereby allowing for competition to exist among the targets from both samples for a single array probe.

A 2-color dual probe sandwich assay can in principle be performed by a number of different methods. As discussed further below, not all are equally suited for use with standard DNA or RNA targets. In the first method, each differentially labeled label probe is first separately hybridized to one of the two target samples (the “label pre-hybridization”). Both samples are then combined and the mixture is hybridized to an array probe. The ratios of the two targets is then determined by detecting the relative amounts of each label probe that has been pre-hybridized to the now array probe-bound targets.

In a second method of performing a 2-color dual probe assay, (the “array pre-hybridization”) the target samples are first hybridized to the array probes, followed by the hybridization of the label probes.

In a third method of performing the hybridization, all elements are present at the same time (the “simultaneous hybridization”). The hybridization of the label probes to the targets, and the targets to the array probes occur in an uncontrolled manner determined by the kinetics and thermodynamics of the system.

For the “label pre-hybridization” method to be successful, it is essential that the pre-hybridized label probes to not dissociate from one target and rebind to the other target after the two samples have been mixed together. If this occurs, the uniqueness of the target labeling is lost, and accurate ratios cannot be determined. This phenomenon can seriously limit the utility of a “label pre-hybridization” approach for performing a 2-color dual probe sandwich assay. Both the “probe pre-hybridization” and “simultaneous hybridization” methods can present serious problems when used with standard DNA or RNA targets. After the two samples are mixed together, the two samples cannot be distinguished, unless each sample has been uniquely tagged with a common primer region, as further discussed below.

An important attribute of dual-probe sandwich assays is that they can impart an additional degree of specificity to the overall measurement. For example, in a dual-probe system where one hybridization event between the target and an array probe defines a spatial location of the target and a second hybridization event between the target and a labeling probe defines the target's presence, the degree to which any mis-hybridization between an array probe and an incorrect target is detected is substantially reduced as long as the number of target species (e.g. targets corresponding to different genes within the sample) that are being interrogated is substantially less than the total number of targets present in the target mixture. Importantly however, it is not possible, using natural nucleic acids alone, to maintain both a two-color and dual-probe sandwich assay that preserves the added specificity advantage described above. This is because traditional sandwich assays rely upon the introduction of a sample-unique yet target species-common labeling probe hybridization site into the target molecules. Thus, all of the targets derived from a given sample will possess a common labeling probe site thereby eliminating any potential for additional specificity coming from the hybridization between the target and labeling probe. Importantly however, the unique UNA chemistries of present invention allow for a combined two-color sandwich assay that preserves the specificity advantage. Whereas standard nucleic acid chemistries allow for this high specificity assay only with a label pre-hybridization technique, UNA chemistries allow label-prehybridization, array pre-hybridization, or simultaneous hybridization sandwich assay techniques.

Of course, those skilled in the art will appreciate that many different array probes corresponding to different targets within the different sample mixtures can be attached to a particular array, each probe being attached to a different region, or feature, of the array. Moreover, any number of samples for which a mixture of UNA targets exist may be assayed on a given array, so long as the targets for each given sample mixture has a different UNA chemistry that can either hybridize to a sample-specific array probe or hybridize to a common array probe where sample discrimination is determined by labeling probes

The assay of the present invention includes UNAs that hybridize with specific base pairing chemistries. According to the present invention, pairs of UNA target samples and UNA probes are provided, which have specific hybridization specificities that are utilized to identify a sample, or expression of a particular gene in the sample. Below we provide some general non-limiting teachings on UNAs and their properties, how target and probe UNAs can be synthesized, methods of creating microarrays that contain UNA probes, and processes for hybridizing and detecting target and probe UNAs.

UNA Properties

In accordance with the present invention, UNAs are produced such that specific binding between a chosen UNA probe and a chosen UNA target sample occur in the presence of other UNA probes and UNA target samples. Therefore, a UNA probe is matched in its UNA base content to a particular UNA target sample and is not matched to other UNA samples. Nucleotides that produce disfavored primer/sample pairs are selected such that a first nucleotide base is not capable of forming a stable base pair with a nucleotide complement. The two complementary nucleotides may have one naturally occurring base and one base analog or may have two base analogs. Disfavored probe/sample pairs that are unable to form stable base pairs have reduced levels of intermolecular base pairing based on UNA content of the probe and the target sample.

UNAs may contain a mixture of nucleotide analogs and naturally occurring nucleotides. UNAs of the present invention may also contain only nucleotide base analogs. More specifically, in accordance with the base pairing formulas outlined in Formula 1 and 2, nucleotides of the first group (A′, T′, G′, C′) and nucleotides of the second group (A*, T*, G*, and C*) may include combinations of natural bases and modified bases or include all modified bases. For example, A′ and T′, which does not form a stable base pair, may be comprised of one nucleotide base analog (A′) and one natural nucleotide (T′). Alternatively, A′ and T′ may be comprised of two nucleotide base analogs. Nucleotide pairs from the second group (e.g. A* and T*) may or may not form stable base pairs (A*=T*or A*≠T*).

UNAs may contain both A′/T* base pair analogs that form stable base pairs and G/C base pairs that form stable base pairs. Alternatively, UNAs may contain G′/C* base pair analogs that form stable base pairs and A/T base pairs that form stable base pairs. UNAs may also contain both sets of analogs that form stable base pairs (A′=T* and G′=C*). For the present invention, nucleotides from the first and second class (e.g. A′, A*) may be mixed in the same molecule.

UNA Target Synthesis

UNA targets can be synthesized by any of a number of methods for use in the present invention. Indeed, any method available in the art may be utilized to generate the UNA targets described herein. In preferred embodiments target UNAs are synthesized by enzymatic methods. For example, target UNAs may be synthesized by template dependent RNA or DNA polymerization.

Polymerization methodologies that utilize template dependent DNA or RNA polymerases are preferred methods for copying genetic material of unknown sequence from biological sources for subsequent sequence and expression analyses. Thus UNAs, which are produced preferably by enzymatic methods, are well suited for generating oligonucleotides and polynucleotides for subsequent hybridization. Moreover, since preferred UNAs are synthesized using DNA and RNA polymerases, UNAs may be synthesized having lengths ranging from several nucleotides to several thousand nucleotides.

Any enzyme capable of incorporating naturally occurring nucleotides, nucleotides base analogs, or combinations thereof into a polynucleotide may be utilized in accordance with the present invention. As examples without limitation, the enzyme can be a primer/DNA template dependent DNA polymerase, a primer/RNA template dependent reverse transcriptase or a promoter-dependent RNA polymerase. Non-limiting examples of DNA polymerases include E. coli DNA polymerase T, E. coli DNA polymerase I Large Fragment (Klenow fragment), or phage T7 DNA polymerase. The polymerase can be a thermophilic polymerase such as Thermus aquaticus (Taq) DNA polymerase, Thermus flavus (Tfl) DNA polymerase, Thermus Thermophilus (Tth) DNA polymerase, Thermococcus litoralis (Tli) DNA polymerase, Pyrococcus furiosus (Pfu) DNA polymerase, Vent™ DNA polymerase, or Bacillus stearothermophilus (Bst) DNA polymerase. Non-limiting examples of reverse transcriptases include AMV Reverse Transcriptase, MMLV Reverse Transcriptase and HIV-1 reverse transcriptase. Non-limiting examples of RNA polymerases suitable for generating RNA version of UNAs include the bacteriophage RNA polymerases from SP6, T7 and T3. Furthermore, any molecule capable of using a DNA or an RNA molecule as a template to synthesize another DNA or RNA molecule can be used in accordance with the present invention. (e.g., self-replicating RNA).

Primer/DNA template-dependent DNA polymerases, primer/RNA template-dependent reverse transcriptases and promoter-dependent RNA polymerases incorporate nucleotide triphosphates into the growing polynucleotide chain according to the standard Watson and Crick base-pairing interactions (see for example; Johnson, Annual Review in Biochemistry, 62; 685-713 (1993), Goodman et al., Critical Review in Biochemistry and Molecular Biology, 28; 83-126 (1993) and Chamberlin and Ryan, The Enzymes, ed. Boyer, Academic Press, New York, (1982) pp 87-108). Some primer/DNA template dependent DNA polymerases and primer/RNA template dependent reverse transcriptases are capable of incorporating non-naturally occurring triphosphates into polynucleotide chains when the correct complementary nucleotide is present in the template sequence. For example, Klenow fragment and AMV reverse transcriptase are capable of incorporating the base analogue iso-guanosine opposite iso-cytidine residues in the template sequence (Switzer et al., Biochemistry 32; 10489-10496 (1993). Similarly, Klenow fragment and HIV-l reverse transcriptase are capable of incorporating the base analogue 2,4-diaminopyrimidine opposite xanthosine in a template sequence (Lutz et al., Nucleic Acids Research 24; 1308-1313 (1996)).

UNAs may also be generated using one of a number of different methods known in the art. These include but are not limited to nick translation for generating labeled target molecules (Feinberg and Vogelstein, Analytical Biochemistry, 132; 6-13 (1983) and Feinberg and Vogelstein, Analytical Biochemistry, 137; 266-267 (1984)), asymmetric PCR methods (Gyllensten and Erlich, Proc. Natl. Acad. Sci. USA. 85; 7652-7656(1988)) that utilize a single primer or a primer having some chemical modification that results in the synthesis of strands of unequal lengths (Williams and Bartel, Nucleic Acids Research, 23; 4220-4221 (1995) and affinity purification methods that utilize either magnetic beads (Hultman et al., Nucleic Acids Research, 17; 4937-4946 (1989)) or streptavidin induced electrophoretic mobility shifts (Nikos, Nucleic Acids Research, 24; 3645-3646 (1996)).

The asymmetric PCR method would be performed using a single target-specific primer and either a single-stranded or double stranded DNA template in the presence of a thermophilic DNA polymerase or reverse transcriptase and the appropriate UNA nucleotide triphosphates. The reaction mixture would be subjected to temperature cycle a defined number of times depending upon the degree of amplification desired. The limitation of the amplification to this type of linear mode is inherent to the designed base-pairing properties of UNAs. Unlike nucleic acids generated from the four standard nucleotides, the UNA replication products are generated from non-complementary pairs of nucleotides and thus cannot serve as templates for subsequent replication events. However the invention does not preclude the use of PCR to amplify the target prior to generation of UNAs by the methods described herein.

UNAs can also be generated using a polymerase extension reaction followed by a strand-selective exonuclease digestion (Little et al., J. Biol Chem. 242, 672 (1967) and Higuchi and Ochamn, Nucleic Acids Research, 17; 5865-(1989)). For example, a target-specific primer is extended in an isothermal reaction using a DNA polymerase or reverse transcriptase in the presence of the appropriate UNA nucleotide triphosphates and a 5′-phosphorylated DNA template. The DNA template strand of the resulting duplex is then specifically degraded using the 5′-phosphorly-specific lambda exonuclease. A kit for performing the latter step is the Strandase Kit™ currently marketed by Novagen (Madison, Wis.).

Single-stranded ribonucleotide (RNA) versions of UNAs can be synthesized using in vitro transcription methods which utilize phage promoter-specific RNA polymerases such as SP6 RNA polymerase, T7 RNA polymerase, and T3 RNA polymerase (see for example Chamberlin and Ryan, The Enzymes, ed. Boyer, Qacademic Press, New York, (1982) pp87-108 and Melton et al., Nucleic Acids Research, 12; 7035 (1984)). For these methods, a double stranded DNA corresponding to the target sequence is generated using PCR methods known in the art in which a phage promoter sequence is incorporated upstream of the target sequence. This double-stranded DNA is then used as the template in an in vitro transcription reaction containing the appropriate phage polymerase and the ribonucleotide triphosphate UNA analogues. Alternatively, a single stranded DNA template prepared according to the method of Milligan and Uhlenbeck, (Methods in Enzymology, 180A, 51-62 (1989)) can be used to generate RNA versions of UNAs having any sequence. A benefit of these types of in vitro transcription methods is that they can result in a 100 to 500-fold amplification of the template sequence.

Structural Modifications to Nucleotides

Nucleotide base analogues having fewer structural changes can also be efficient substrates for DNA polymerase reactions. For example, a number of polymerases can specifically incorporate inosine across cytidine residues (Mizusawa et al., Nucleic Acids Research, 14; 1319 (1986). The analogue 2-aminoadenosine triphosphate can also be efficiently incorporated by a number of DNA polymerases and reverse transcriptases (Bailly and Waring, Nucleic Acids Research, 23; 885 (1996). In fact, 2-aminoadenosine is a natural substitute for adenosine in S-2L cyanophage genomic DNA. However, for the present invention 2-aminoadenosine is defined as a non-naturally occurring base. The 2-aminoadenosine ribonucleotide-5′-triphosphate is a good substrate for E. coli RNA polymerase (Rackwitz and Scheit, Eur. J. Biochem., 72, 191 (1977)). The adenosine analogue 2-aminopurine can also be efficiently incorporated opposite T residues by E. coli DNA polymerase (Bloom et al., Biochemistry 32; 11247-11258 (1993) but can mispair with cytidine residues as well (see Law et al., Biochemistry 35; 12329-12337 (1996)).

Any structural modifications to a nucleotide that do not inhibit the ability of an enzyme to incorporate the nucleotide analogue may be used in the present invention if the modifications do not result in a violation of the base pairing rules set forth in the present invention. Modifications include but are not limited to structural changes to the base moiety (e.g. C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine), changes to the ribose ring (e.g. 2′-hydroxyl, 2′-fluro), and changes to the phosphodiester linkage (e.g. phosphorothioates and 5′-N-phosphoamidite linkages).

Watson-Crick base-pairing schemes can accommodate a number of modifications to the ribose ring, the phosphate backbone and the nucleotide bases (Saenger, Principles of Nucleic Acid Structure, Springer-Verlag, New York, N.Y. 1983). Certain modified bases such as inosine, 7-deazaadenosine, 7-deazaguanosine and deoxyuridine decrease the stability of base-pairing interactions when incorporated into polynucleotides. The dNTP forms of these modified nucleotides are efficient substrates for DNA polymerases and have been used to reduce sequencing artifacts that result from target and extension product secondary structures (Mizusawa et al., Nucleic Acids Research, 14; 1319. 1986). Other modified nucleotides, such as 5-methylcytidine, C-5 propynyl-cytidine, C-5 propynyl-uridine and 2-aminoadenosine increase the stability of duplex when incorporated into polynucleotides (Wagner et al., Science, 260; 1510. 1993) and have been used to increase the hybridization efficiency between oligonucleotide probes and target sequences.

2-Anminoadenosine (D), 2-Thiothymidine (2-thioT)

According to the invention, probes are generated that bind to a chosen UNA target sample, and not to other UNA target samples based on their UNA content. Without being limited by theory, in one example a D/2-thioT base pair analog is prevented from forming a stable base pair presumably due to a steric clash between the thio group of 2-thioT and the exocyclic amino group of 2-aminoadenosine as a result of the larger atomic radius of the sulfur atom (see FIG. 1). This tilts the nucleotide bases relative to one another such that only one hydrogen bond is able to form. It is also known that thionyl sulfur atoms are poorer hydrogen-bonding acceptors than carbonyl oxygen atoms, which could also contribute to the weakening of the D/2-thioT base pair.

In designing a probe that is able to bind a particular UNA target sample, other base pairs are highly favored. For example, the 2-aminoadenosine (D) is capable of forming a stable base-pair with thymidine (T) through three hydrogen bonds in which a third hydrogen bonding interaction is formed between the 2-amino group and the C2 carbonyl group of thymine (FIG. 1). As a result, the D/T base pair is more stable thermodynamically than an A/T base pair. In addition, 2-thiothymidine (2-thioT) is capable of forming a stable hydrogen bonded base pair with adenosine (A), which lacks an exocyclic C2 group to clash with the 2-thio group.

Therefore, polynucleotide molecules with 2-aminoadenosine (D) and 2-thioT replacing A and T respectively have a reduced ability to form D/2-thioT base pairs but are still capable of hybridizing to polynucleotides of substantially complementary sequence comprising A and T and lacking D and 2-thioT. Without being limited by theory, the aforementioned proposed mechanisms regarding the factors responsible for stabilizing and disrupting the A/T and G/C analogue pairs are not meant in any way to limit the scope of the present invention and are valid irrespective of the nature of the specific mechanisms.

Gamper and coworkers (Kutyavin et al. Biochemistry, (1996) 35: 11170- 11176) determined experimentally that short oligonucleotide duplexes containing D/T base pairs that replace A/T base pairs have melting temperatures (Tm) as much as 10° C. higher than duplexes of identical sequence composed of the four natural nucleotides. This is due mainly to the extra hydrogen bond provide by the 2-amino group. However, the duplexes designed to form opposing D/2-thioT base pairs exhibited Tms as much as 25° C. lower than the duplex of identical sequence composed of standard A/T base pairs. The authors speculate that this is mainly due to the steric clash between the 2-thio group and the 2-amino group, which destabilizes the duplex. Although the base-pairing selectivity for these analog pairs has been experimentally tested for only DNA duplexes, it is likely that these same rules will hold for RNA duplexes and DNA/RNA heteroduplexes as well. This would allow for RNA versions of UNAs to be generated by transcription of PCR or cDNA products using the ribonucleotide triphosphate forms of the UNA analog pairs and RNA polymerases (e.g., “cUNAs”).

Inosine (I) and Pyrrolo-pyrimnidine (P)

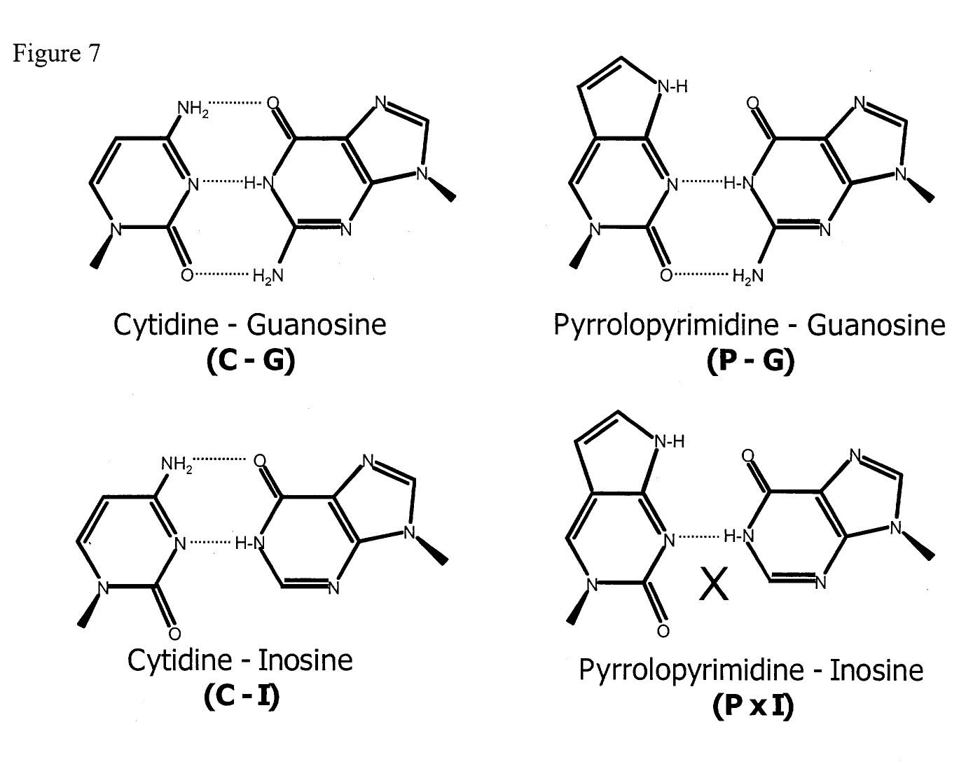

The inosine (I) and pyrrolo-pyrimidine (P) I/P base pair analog is also depicted in FIG. 7. Inosine, which lacks the exocyclic 2-amino group of guanine, forms a stable base pair with cytosine through two hydrogen bonds (vs. three for G/C). The other member of the I/P analog is pyrrolo-pyrimidine (P), which is capable of forming a stable base pair with guanine despite the loss of the 4-amino hydrogen bond donor of cytosine. FIG. 7 shows that a P/G base pair is also formed through two hydrogen bonds. The N7 group of P is spatially confined by the pyrrole ring and is unable to form a hydrogen bond with the C6 carbonyl O of guanine. However, this does not prevent the formation of the other two hydrogen bonds between P/G. The I/P base pair is only capable of forming one hydrogen bond (as depicted in FIG. 7) and is therefore not a stable base pair. As a result, polynucleotide molecules with I and P replacing G and C respectively have a reduced ability to form I/P base pairs but are still capable of hybridizing to polynucleotides of substantially complementary sequence comprising G and C and lacking I and P.

Woo and co-workers (Woo et al., Nucleic Acids Research, 24; 2470 (1996)) showed that introducing either P or I into 28-mer duplexes to form P/G and I/C base-pairs decreased the Tm of the duplex by -0.5 and -1.9° C. respectively per modified base-pair. These values reflect the slight destabilization attributable to the G/P pair and a larger destabilization due to the I/C pair. However, introducing P and I into the duplexes such that opposing I/P base pairs are formed reduced the Tm by -3.3° C. per modified base pair. Therefore the I/P base pairs are more destabilizing.

2-Aminoadenosine (D), 2-Thiothymidine (2-thioT), Inosine (I) & Pyrrolo-pyrimidine (P)

In a particularly preferred embodiment, the nucleotide analogs 2-aminoadenosine (D), 2-thiothymidine (2-thioT), inosine (I) and pyrrolo-pyrimidine (P) are used to generate UNA probes and UNA target samples that retain their ability to hybridize to a complementary strand through Watson-Crick base-pairs. The structures of the D=2-thioT, I=P and the natural base pairs along with various combinations of the natural and base analogs are shown in FIGS. 1 and 7.

UNAs comprising D, 2-thioT, I, and P

In accordance with the present invention, nucleic acid molecules (UNAs) are generated by performing primer dependent, template directed polymerase reactions using the nucleotide 5′-triphosphate forms of the appropriate analog pairs. These include, e.g., 2-amino-2′-deoxyadenosine-5′-triphosphate (dDTP), 2-thio-2′deoxythymidine-5′-triphosphate (d-2-thioTTP), 2′-deoxyinosine-5′-triphosphate (dITP) and 2′-deoxypyrrolo-pyrimidine-5′-triphosphate (dPTP).

UNAs comprising D, 2-thioT, 2-thioC, and G

In yet another preferred embodiment of the present invention, the nucleotide base pair analogs 2-aminoadenosine/2-thiothymidine (D/2-thioT) and 2-thiocytidine/guanosine (2-thioC/G) are used in primer dependent polymerase reactions to generate nucleic acid molecules that retain their ability to form Watson-Crick base pairs with oligonucleotides composed of the four natural bases. 2-thioC and G are unable to form a stable base pair (FIG. 2). The presence of a 2-thiocarbonyl group in cytosine replacing the C2 carbonyl group effectively removes the hydrogen bond acceptor at that position and causes a steric clash due to the large ionic radius of sulfur as compared to oxygen. As a result, 2-thioC/G is only capable of forming a single hydrogen bond and is thus not a stable base pair. However, 2-thioC and I are capable of forming a stable base pair through two hydrogen bonds since the removal of the 2-amino exocyclic group of guanine that results in inosine effectively removes the steric clash between the C2 sulfur of 2-thioC and the 2-amino group of guanine.

Therefore, UNAs of the present invention may be generated enzymatically using the 5′-triphosphate forms of the base pair analogs. These include; 2-amino-2′-deoxyadenosine- 5′-triphosphate (dDTP), 2-thio-2′-deoxythymidine-5′-triphosphate (d-2-thioTTP), 2′-deoxyguanosine-5′-triphosphate (dGTP) and 2-thio-2′-deoxycytidine-5′-triphosphate (d-2-thioCTP). For example, since 2-aminoadenosine, 2thiothymidine, 2-thiocytidine and guanosine are still capable of forming stable base pairs with thymidine, adenosine, inosine and cytidine respectively, UNAs comprising (A, T, 2-thioC, G) or (D, 2-thioT, 2-thioC, G) should be able to specifically hybridize to oligonucleotides composed of the appropriate bases according to the base pairing rules discussed.

The 2-thioC/G base pair analog provides an example of a base pair analog comprising a natural nucleotide base and a nucleotide base analog, which cannot form a stable base pair. As previously stated, polynucleotides containing 2-thiocytidine and guanosine can form base pairs with polynucleotides of substantially complementary sequences through 2-thioC/I and C/G base pairs. Therefore, UNAs comprising 2-thioC/G are capable of hybridizing to polynucleotide molecules also containing base analogs (inosine).

UNA Probe and Array Synthesis

UNA probes of the present invention are synthesized and attached to solid supports, e.g., microarrays, for detection and identification of target UNA samples. Probes that hybridize to target UNAs can be synthesized enzymatically, as described above, or chemically, as long as the method of synthesis is compatible with the base pairing chemistry of the UNA nucleotides. For example, for enzyme synthesis, the enzymes are able to incorporate the chosen UNA nucleotides into the UNA probe. For chemical synthesis, the chemicals are compatible with UNA chemistry, for example, able to form UNA phosphoramides etc.

In certain preferred embodiments, enzymatic methods are preferred for the synthesis of long UNA probes. For example, probes over 100 nucleotides, such as the UNA probes, are used as surface-bound probes on the array. Longer probes also include cDNA probes that contain UNA bases, e.g., “cUNA” probes, as described above. In other preferred embodiments, shorter probes, for example, less than 100 nucleotides, are synthesized by chemical synthesis methods. Such shorter probes are preferred for use in the sandwich-type assays that utilize a single probe on an array and a secondary labeled probe for detection, described above.

Oligonucleotide probes may be synthesized, in situ, on an array or bead surface in either the 3′ to 5′ or 5′ to 3′ direction using the 3′ -β-cyanoethyl-phosphoramidites or 5′-β-cyanoethyl-phosphoramidites and related chemistries known in the art. In situ synthesis of the oligonucleotides can be performed in the 5′to 3′ direction using nucleotide-coupling chemistries that utilize 3′-photoremovable protecting groups (U.S. Pat. No. 5,908,926). Alternatively, the oligonucleotide probes may be synthesized on the standard control pore glass (CPG) in the more conventional 3′to 5′direction using the standard 3′-p-cyanoethyl-phosphoramidites and related chemistries (Caruthers M. et al., Method Enzymol, 154; 287-313 (1987), Caruthers Science 230:281-285(1985); Itakura et al., Ann. Rev. Biochem. 53: 323-356 (1984), Hunkapillar et al. Nature 310: 105-110 (1984); and in Synthesis of Oligonucleotide Derivatives in Design and Targeted Reaction of Oligonucleotide Derivatives, CRC Press, Boca Raton, Fla., pages 100 et seq.; U.S. Pat. No. 4,458,066; U.S. Pat. No. 4,500,707; U.S. Pat. No. 5,153,319; U.S. Pat. No. 5,869,643; and EP 0294196, incorporated by reference herein in their entirety) and incorporating a primary amine or thiol functional group onto the 5′ terminus of the oligonucleotide (Sproat et al., Nucl. Acids Res, 15, 4837(1987); and Connolly and Rider, Nucl. Acids Res, 13, 4485 (1985)). The oligonucleotides may then be covalently attached to an array or bead surface via their 5′ termini using thiol or amine-dependent coupling chemistries known in the art. The density of the probes on the array surface can range from about 1,000 to 200,000 probe molecules per square micron. The probe density can be controlled by adjusting the density of the reactive groups on the surface of the electrode for either the in situ synthesis post-synthesis deposition methods.

One feature of the subject invention is the array of UNA probes arranged on a support in a pattern at least according to which gene the UNA probes hybridize to, e.g., arranged in pairs of UNA probes capable of hybridizing to the same gene in different samples (see, e.g., U.S. Pat. No. 6,287,768, incorporated herein by reference). The UNA probes of the subject arrays are typically UNA base containing nucleic acids or at least mimetics or analogues of naturally occurring polymeric compounds. Biopolymeric compounds of particular interest are ribonucleic acids, as well as deoxyribonucleic acid derivatives thereof, generated through a variety of processes (usually enzymatic processes) such as reverse transcription, etc., e.g. cDNA amplified from RNA (both single and double stranded), cDNA inserts from cDNA libraries, and the like. Of course, those skilled in the art will recognize that any of these processes may be carried out using one or more UNA bases.

The probe, e.g. RNA, may be amplified, e.g. by PCR transcription in the presence of a UNA base containing reaction mixture so that the naturally occurring bases are replaced with the desired UNA bases.

The UNA probes of the invention are capable of hybridizing to genes from target samples that also contain UNA bases. In certain preferred embodiments, like the UNA probes, the target samples are ribonucleic acids. Ribonucleic acids of interest that are target samples, according to the invention, include target samples derived from total RNA, polyA^{+RNA}, polyA^{-RNA}, snRNA (small nuclear), hnRNA (heterogeneous nuclear), cytoplasmic RNA, pre mRNA, mRNA, cRNA (complementary), and the like. The initial RNA, e.g., mRNA, may be present in a variety of different samples, where the sample will typically be derived from (“derived from” meaning amplified from a sample from nature) a physiological source. The physiological source may be a variety of eukaryotic sources, with physiological sources of interest including sources derived from single-celled organisms such as yeast and multicellular organisms, including plants and animals, where the physiological sources from multicellular organisms may be derived from particular organs or tissues of the multicellular organism, or from isolated cells derived therefrom. For example, the target samples may be based on genes obtained or derived from naturally occurring biological sources, particularly mammalian sources and more particularly mouse, rat or human sources, where such sources include: fetal tissues, such as whole fetus or subsections thereof, e.g. fetal brain or subsections thereof, fetal heart, fetal kidney, fetal liver, fetal lung, fetal spleen, fetal thymus, fetal intestine, fetal bone marrow; adult tissues, such as whole brain and subsections thereof, e.g. amygdala, caudate nucleus, corpus callosum, hippocampus, hypothalamus, substantia nigra, subthalamic nucleus, thalamus, cerebellum, cerebral cortex, medula oblongata, occipital pole, frontal lobe, temporal lobe, putamen, adrenal cortex, adrenal medula, nucleus accumbens, pituitary gland, adrenal gland and subsections thereof, such as the adrenal cortex and adrenal medulla, aorta, appendix, bladder, bone marrow, colon, colon proximal with out mucosa, heart, kidney, liver, lung, lymph node, mammary gland, ovary, pancreas, peripheral leukocytes, placental, prostate, retina, salivary gland, small intestine, skeletal muscle, skin, spinal cord, spleen, stomach, testis, thymus, thyroid gland, trachae, uterus, uterus without endometrium; cell lines, such as breast carcinoma T-47D, colorectal adenocarcinoma SW480, HeLa, leukemia chronic myelogenous K-562, leukemia lymphoblastic MOLT-4, leukemia promyelocytic HL-60, lung carcinoma A549, lumphoma Burkitt's Daudi, Lymphoma Burkitt's Raji, Melanoma G361, teratocarcinoma PA-1, leukemia Jurkat; and the like. Where the target samples are derived from naturally occurring sources, such as mammalian tissues as described above, the target samples may be derived from the same or different organisms, but will usually be derived from the same organism. In addition, the target samples arrayed on the plate can be derived from normal and disease or condition states of the same organism, like cancer, stroke, heart failure; aging, infectious diseases, inflammation, exposure to toxic, drug or other agents, conditional treatment, such as heat shock, sleep deprivation, physical activity, etc., different developmental stages, and the like. Of course, the target samples of the invention, although encoding genes derived from various sources, have been synthesized so that they contain UNA bases.

In obtaining the sample of RNA to be analyzed from the physiological source from which it is derived, the physiological source may be subjected to a number of different processing steps, where such processing steps might include tissue homogenization, cell isolation and cytoplasm extraction, nucleic acid extraction and the like, where such processing steps are known to those of skill in the art. Methods of isolating RNA from cell, tissues, organs, or whole organisms are known to those of skill in the art and are described in Maniatis et al. (1989), Molecular Cloning: A Laboratory Manual 2 d Ed. (Cold Spring Harbor Press).

In the subject arrays, the UNA probes are preferably stably associated with the surface of a support. Enzymatically or chemically synthesized probes may be deposited on the surface. Chemically synthesized UNA probes may be synthesized in situ on the array surface (see, for example, U.S. Pat. No. 5,474,796; U.S. Pat. No. 5,510,270; U.S. Pat. No. 5,552,270; and U.S. Pat. No. 5,554,501, each of which is incorporated herein by reference).

By stably associated is meant that the UNA probes maintain their position relative to the support under hybridization and washing conditions. As such, the UNA probes can be non-covalently or covalently stably associated with the support surface. Examples of non-covalent association include non-specific adsorption, specific binding through a specific binding pair member covalently attached to the support surface, and entrapment in a matrix material, e.g. a hydrated or dried separation medium, which presents the UNA probe in a manner sufficient for binding, e.g. hybridization, to occur. Examples of covalent binding include covalent bonds formed between the UNA probe and a functional group present on the surface of the support, e.g. —OH, where the functional group may be naturally occurring or present as a member of an introduced linking group, as described in greater detail below.

As mentioned above, the array is typically present on a substrate. Certain substrates are rigid meaning that the support is solid and does not readily bend, i.e. the support is not flexible. Examples of solid materials, which are not rigid supports with respect to the present invention, include membranes, flexible plastic films, and the like. As such, rigid substrates are sufficient to provide physical support and structure to the UNA probes present thereon under the assay conditions in which the array is employed, particularly under high throughput handling conditions.

The substrates upon which the subject patterns of UNA probes are preferably presented in the subject arrays may take a variety of configurations ranging from simple to complex, depending on the intended use of the array. Thus, the substrate could have an overall slide or plate configuration, such as a rectangular or disc configuration, where an overall rectangular configuration, as found in standard microtiter plates and microscope slides, is preferred. For example, the length of the substrates may be at least about 1 cm and may be as great as 40 cm or more, but usually does not exceed about 30 cm and may often not exceed about 15 cm. The width of substrate may be at least about 1 cm and may be as great as 30 cm, but usually does not exceed 20 cm and often does not exceed 10 cm. The height of the substrate will generally range from 0.01 mm to 10 mm, depending at least in part on the material from which the substrate is fabricated and the thickness of the material required to provide the requisite rigidity.

The substrates of the subject arrays may be fabricated from a variety of materials. The materials from which the substrate is fabricated should ideally exhibit a low level of non-specific binding of target sample during hybridization or specific binding events. In many situations, it will also be preferable to employ a material that is transparent to visible and/or UV light. Specific materials of interest include: glass; plastics, e.g. polytetrafluoroethylene, polypropylene, polystyrene, polycarbonate, and blends thereof, and the like; metals, e.g. gold, platinum, and the like; etc.

The substrate of the subject arrays comprise at least one surface on which a pattern of UNA probe molecules is present, where the surface may be smooth or substantially planar, or have irregularities, such as depressions or elevations. The surface on which the pattern of UNA probes is presented may be modified with one or more different layers of compounds that serve to modulate the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like. Polymeric layers of interest include layers of: peptides, proteins, polynucleic acids or mimetics thereof, e.g. peptide nucleic acids and the like; polysaccharides, phospholipids, polyurethanes, polyesters, polycarbonates, polyureas, polyamides, polyethyleneamines, polyarylene sulfides, polysiloxanes, polyimides, polyacetates, and the like, where the polymers may be hetero- or homopolymeric, and may or may not have separate functional moieties attached thereto, e.g. conjugated.

The concentration of the UNA probe positions on the surface of the support is selected to provide for adequate sensitivity of binding events with a target sample, where the concentration will generally range from about 1 to 5, usually from about 2 to 25 and more usually from about 5 to 15 ng/mm². As summarized above, the subject arrays comprise a plurality of different UNA probe pairs or sets of UNA probes, where the number of UNA probes is at least 2. In some embodiments, the arrays have at least 10 distinct spots, usually at least about 20 distinct spots, and more usually at least about 50 distinct spots, where the number of spots may be as high as 10,000 or higher. The arrays of the subject invention may be used directly in binding assays, i.e., hybridization assays, using well known technologies, e.g. contacting with target sample in a suitable container, under a coverslip, etc, or may be incorporated into a structure that provides for ease of analysis, high throughput, or other advantages, such as in a biochip format, a multiwell format and the like. For example, the subject arrays could be incorporated into a biochip type device in which one has a substantially rectangular shaped cartridge comprising fluid entry and exit ports and a space bounded on the top and bottom by substantially planar rectangular surfaces, wherein the array is present on one of the top and bottom surfaces.

Alternatively, the subject arrays may be incorporated into a high throughput or multiwell device, wherein each array is bounded by raised walls in a manner sufficient to form a reaction container wherein the array is the bottom surface of the container. Such high throughput devices are described in U.S. patent application Ser. No. 08/974,298, now abandoned, the disclosure of which is herein incorporated by reference. Generally in such devices, the devices comprise a plurality of reaction chambers, each of which contains the array on the bottom surface of the reaction chamber. By plurality is meant at least 2, usually at least 4 and more usually at least 24, where the number of reaction chambers may be as high as 96 or higher, but will usually not exceed 100. The volume of each reaction chamber may be as small as 10 μl but will usually not exceed 500 μl.

The subject arrays may be prepared as follows. The substrate or support can be fabricated according to known procedures, where the particular means of fabricating the support will necessarily depend on the material from which it is made. For example, with polymeric materials, the support may be injection molded, while for metallic materials, micromachining may be the method of choice. Alternatively, supports such as glass, plastic, or metal sheets can be purchased from a variety of commercial sources and used. The surface of the support may be modified to comprise one or more surface modification layers, as described above, using standard deposition techniques.

Typically, the next step in the preparation process is to prepare the pattern of UNA probe molecules and then stably associate the UNA probe molecules with the surface of the support. The complex original source of UNA probe molecules may be obtained from (e.g., amplified from) its naturally occurring physiological source using standard techniques. Protocols for isolating nucleic acids are described in: Maniatis et al., Molecular Cloning: A Laboratory Manual (Cold Spring Harbor Press)(1989), incorporated herein by reference. Such methods typically involve subjection of the original biological source to one or more of tissue/cell homogenization, nucleic acid extraction, chromatography, centrifugation, affinity binding and the like. UNA probe molecule preparation can further include one or more treatments such as: reverse transcription; nuclease treatment; protease digestion; in vitro transcription; DNA amplification; enzymatic or chemical modification of RNA, such as the introduction of functional moieties, such as biotin, digoxigenin, fluorescent moieties, antigens, chelator groups, chemically active or photoactive groups, etc.; and the like.

The UNA probes may be deposited on the support surface using any convenient means, such as by using an “ink-jet” device, mechanical deposition, pipetting and the like. After deposition of material onto the solid surface, it can be treated in different ways to provide for stable association of the UNA probe, blockage of non-specific binding sites, removal of unbound UNA probe, and the like.

The UNA probes can also be formed by direct chemical synthesis and deposited on the support surface, or by in situ synthesis methods as previously described.

Following stable placement of the pattern of UNA probe molecules on the support surface, the resultant array may be used as is or incorporated into a biochip, multiwell or other device, as describe above, for use in a variety of binding applications.

The subject arrays or devices into which they are incorporated may conveniently be stored following fabrication for use at a later time. Under appropriate conditions, the subject arrays are capable of being stored for at least about 6 months and may be stored for up to one year or longer. The subject arrays are generally stored at temperatures between about −20° C. to room temperature, where the arrays are preferably sealed in a plastic container, e.g. bag, and shielded from light.

Applications in which the subject arrays find particular use are expression analysis applications. Such applications generally involve the following steps: (a) preparation of a target sample; (b) contact of the target sample with the array under conditions sufficient for the target sample to bind with corresponding UNA probe, e.g. by hybridization or specific binding; (c) removal of unbound target from the array by washing the array under appropriate stringency; and (d) detection of bound target sample. Each of these steps will be described in greater detail below.

How the target sample is prepared will necessarily depend on the specific nature of the target sample. For nucleic acid samples, the samples may be ribo- or deoxyribonucleotides, as well as hybridizing analogues or mimetics thereof, e.g. nucleic acids in which the phosphodiester linkage has been replaced with a substitute linkage, such as a phosphorothioate, methylimino, methylphosphonate, phosphoramidite, guanidine and the like; and nucleic acids in which the ribose subunit has been substituted, e.g. hexose phosphodiester, peptide nucleic acids; locked nucleic acids; and the like. The target sample will have sufficient complementarity to a UNA probe to provide for the desired level of sequence specific hybridization.

Hybridizing Conditions and Processing

The next step in the subject method is to contact the target sample with the array under conditions sufficient for binding between the target sample and the UNA probe on the array. For example, where the target sample and UNA probe are nucleic acids, the target sample will be contacted with the array under conditions sufficient for hybridization to occur between the target sample and the UNA probe, where the hybridization conditions will be selected in order to provide for the desired level of hybridization specificity.

Contact of the array and target sample involves contacting the array with an aqueous medium comprising the target sample. Contact may be achieved in a variety of different ways depending on the specific configuration of the array. For example, where the array simply comprises the pattern of UNA probes on the surface of a “plate-like” substrate, contact may be accomplished by simply placing the array in a container comprising the target sample solution, such as a polyethylene bag, small chamber, and the like. In other embodiments where the array is entrapped in a separation media bounded by two plates, the opportunity exists to deliver the target sample via electrophoretic means. Alternatively, where the array is incorporated into a biochip device having fluid entry and exit ports, the target sample solution can be introduced into the chamber in which the pattern of UNA probe molecules is presented through the entry port, where fluid introduction could be performed manually or with an automated device. In multiwell embodiments, the target sample solution will be introduced in the reaction chamber comprising the array, either manually, e.g. with a pipette, or with an automated fluid handling device.

Contact of the target sample solution and the UNA probes will be maintained for a sufficient period of time for binding between the target sample and the UNA probe to occur. Although dependent on the nature of the target sample and UNA probe, contact will generally be maintained for a period of time ranging from about 10 min to 24 hrs, usually from about 30 min to 12 hrs and more usually from about I hr to 6 hrs.

Those skilled in the art will appreciate that the hybridization conditions used in the invention can vary depending on the particular UNA probes and targets contacting one another. In one embodiment of the present invention, hybridization conditions are chosen to allow hybridization of a first UNA probe to a specific nucleic acid sequence or region of a first target sample, whereas a second UNA probe hybridizes to the same nucleic acid sequence or region of a second target sample. Some hybridization conditions are as follows.

The specificity and kinetics of hybridization have been described in detail by, e.g., Wetmur and Davidson J. Mol. Biol., 31:349-370(1968); Britten and Kohne Science 161:529-530 (1968); and Kanehisa, Nuc. Acids Res. 12:203-213(1984); each of which is hereby incorporated herein by reference and are applicable to UNA containing nucleic acids. Parameters which are well known to affect specificity and kinetics of reaction include salt conditions, ionic composition of the solvent, hybridization temperature, length of matching base sequences (e.g., UNA bases that are compatible with each other and will hybridize according to the present invention), guanine and cytosine (GC) content, presence of hybridization accelerators, pH, specific bases found in the matching sequences, solvent conditions, and addition of organic solvents. In particular, the salt conditions required for driving highly mismatched sequences to completion typically include a high salt concentration. The typical salt used is sodium chloride (NaCl), however, other ionic salts may be utilized, e.g., KCl. Depending on the desired stringency hybridization, the salt concentration will often be less than about 3 molar, more often less than 2.5 molar, usually less than about 2 molar, and more usually less than about 1.5 molar. For applications directed towards higher stringency matching, the salt concentrations would typically be lower ordinary high stringency conditions will utilize salt concentration of less than about 1 molar, more often less then about 750 millimolar, usually less than about 500 millimolar, and may be as low as about 250 or 150 millimolar.

The kinetics of hybridization and the stringency of hybridization both depend upon the temperature at which the hybridization is performed and the temperature at which the washing steps are performed. Temperatures at which steps for low stringency hybridization are desired would typically be lower temperatures, e.g., ordinarily at least about 15° C., more ordinarily at least about 20° C., usually at least about 25° C., and more usually at least about 30° C. For those applications requiring high stringency hybridization, or fidelity of hybridization and sequence matching, temperatures at which hybridization and washing steps are performed would typically be high, for example, temperatures in excess of about 35° C. would often be used, more often in excess of about 40° C., usually at least about 45° C., and occasionally even temperatures as high as about 500 C. or 60° C. or more. Of course, even higher temperatures may disrupt the hybridization. Thus, for stripping of targets from substrates, as discussed below, temperatures as high as 80° C., or even higher may be used.

The base composition of the UNA probe and/or target UNA involved in hybridization affects the temperature of melting, and the stability of hybridization as discussed in the above references. However, the bias of GC rich sequences to hybridize faster and retain stability at higher temperatures can be compensated for by the inclusion in the hybridization incubation or wash steps of various buffers. It should also be noted that kinetics and thermodynamic stability of the UNA target-UNA probe hybrids will be effected by the specific UNA nucleotide composition of both the target and probe. For example, it is known that the D=T and 2-thioT=A base-pairs are actually more stable than that of the natural A=T base-pair (see (Kutyavin et al. Biochemistry, (1996) 35: 11170-11176). Sample buffers that can compensate for particular base-pairing stability bias include the triethly and trimethyl ammonium buffers. See, e.g., Wood et al. Proc. Natl. Acad. Sci. USA, 82:1585-1588 (1987); and Khrapko, K. et al. FEBS Letters 256:118-122(1989), incorporated herein by reference.

The rate of hybridization can also be affected by the inclusion of particular hybridization accelerators. These hybridization accelerators include the volume exclusion agents characterized by dextran sulfate, or polyethylene glycol (PEG). Dextran sulfate is typically included at a concentration of between 1% and 40% by weight. The actual concentration selected depends upon the application, but typically a faster hybridization is desired in which the concentration is optimized for the system in question. Dextran sulfate is often included at a concentration of between 0.5% and 2% by weight or dextran sulfate at a concentration between about 0.5% and 5%. Alternatively, proteins that accelerate hybridization may be added, e.g., the recA protein found in E. coli or other homologous proteins.

With respect to those embodiments where specific reagents are not oligonucleotides, the conditions of specific interaction would depend on the affinity of binding between the specific reagent and its target. Typically parameters that would be of particular importance would be pH, salt concentration anion and cation compositions, buffer concentration, organic solvent inclusion, detergent concentration, and inclusion of such reagents such as chaotropic agents. In particular, the affinity of binding may be tested over a variety of conditions by multiple washes and repeat scans or by using reagents with differences in binding affinity to determine which reagents bind or do not bind under the selected binding and washing conditions. The spectrum of binding affinities may provide an additional dimension of information that may be very useful in identification purposes and mapping.

Of course, the specific hybridization conditions will be selected to correspond to a discriminatory condition which provides a positive signal where desired (i.e., when the first probe is hybridized specifically to the first target sample, and the second probe is hybridized specifically to the second target sample) but fails to show a positive signal at affinities where interaction is not desired. This may be determined by a number of titration steps or with a number of controls that will be run during the hybridization and/or washing steps to determine at what point the hybridization conditions have reached the stage of desired specificity.

Following binding of target sample and UNA probe, the resultant hybridization patterns of labeled target sample may be visualized or detected in a variety of ways, with the particular manner of detection being chosen based on the particular label of the nucleic acid, where representative detection means include, e.g., scintillation counting, autoradiography, fluorescence measurement, calorimetric measurement, light emission measurement and the like.

The method may or may not further include a non-bound label removal step prior to the detection step, depending on the particular label employed on the target sample. For example, in homogenous assay formats a detectable signal is only generated upon specific binding of target sample to UNA probe. As such, in homogenous assay formats, the hybridization pattern may be detected without a non-bound label removal step. In other embodiments, the label employed will generate a signal whether or not the target sample is specifically bound to its UNA probe. In such embodiments, the non-bound labeled target sample is removed from the support surface. One means of removing the non-bound labeled target sample is to perform the well known technique of washing, where a variety of wash solutions and protocols for their use in removing non-bound label are known to those of skill in the art and may be used. Alternatively, in those situations where the UNA probes are entrapped in a separation medium in a format suitable for application of an electric field to the medium, the opportunity may arise to remove non-bound labeled target sample from the UNA probe by electrophoretic means.

The above assays can be used to simultaneously determine the expression level of a particular gene in multiple samples. The gene expression level in the particular tissue being analyzed can be derived from the intensity of the detected signal. To ensure that an accurate level of expression is derived, a housekeeping gene of known expression level can also be detected, e.g. using a multiplex approach as described above, to provide for a control signal level in order to calibrate the detected signal.

As such, the subject arrays find use in a variety of different gene expression analysis applications, including differential expression analysis of different samples of diseased and normal tissue, e.g. neoplastic and normal tissue; different tissues or subtypes; tissues and cells under different condition states, like predisposition to disease, age, exposure to pathogens or toxic agents, etc.; and the like.

Also provided are kits for performing binding assays using the subject arrays, where kits for carrying out differential gene expression analysis assays are preferred. Such kits according to the subject invention will at least comprise an array according to the subject invention, where the array may simply comprise a pattern of UNA probe molecules on a planar support or be incorporated into a multiwell configuration, biochip configuration, or other configuration. The kits may further comprise one or more additional reagents for use in the assay to be performed with the array, where such reagents include: target sample generation reagents, e.g. buffers, primers, enzymes, labels and the like; reagents used in the binding step, e.g. hybridization buffers; signal producing system members, e.g. substrates, fluorescent-antibody conjugates, etc.; and the like.

Finally, systems that incorporate the subject arrays, particularly the biochip and multiwell configurations of the subject arrays, are provided, where the systems find use in high throughput gene expression analysis in which information regarding the expression level of a gene in a tissue is desired. By the term “system” is meant the working combination of the enumerated components thereof, which components include those components listed below. Systems of the subject invention will generally include the array of UNA probes, a fluid handling device capable of contacting the target sample fluid and all reagents with the pattern of UNA probe molecules on the array and delivery and removing wash fluid from the array surface; a reader which is capable of providing identification of the location of positive target sample/UNA probe binding events and the intensity of the signal generated by such binding events; and preferably a computer means which capable of controlling the actions of the various elements of the system, i.e. when the reader is activated, when fluid is introduced and the like.

Detection

In certain embodiments, the probe molecule will be labeled to provide for detection in the detection step. By labeled is meant that the probe comprises a member of a signal producing system and is thus detectable, either directly or through combined action with one or more additional members of a signal producing system. Examples of directly detectable labels include isotopic and fluorescent moieties incorporated into, usually covalently bonded to, a moiety of the probe, such as a nucleotide monomeric unit, e.g. dNMP of the primer, or a photoactive or chemically active derivative of a detectable label which can be bound to a functional moiety of the probe molecule. Isotopic moieties or labels of interest include ³²P, ³³P, ³⁵S, ¹²⁵I, and the like. Fluorescent moieties or labels of interest include coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. Texas red, tetramethylrhodamine, eosins and erythrosins, cyanine dyes, e.g. Cy3 and Cy5, macrocyclic chelates of lanthanide ions, e.g. quantum die^{™}, fluorescent energy transfer dyes, such as thiazole orange-ethidium heterodimer, TOTAB, etc. Representative fluorescence detection devices include the Affymetrix GeneArray Scanner (Affymetrix, Santa Clara, Calif.) and Axon GenePix 4000™ microarray scanner (Axon Instruments, Foster City, Calif.). Also of interest are nanometer sized particle labels detectable by light scattering, e.g. “quantum dots.” Labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugate antibody; and the like. Additional labels of interest include those that provide for signal only when the probe with which they are associated is specifically bound to a UNA probe molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076, incorporated herein by reference.

It is also anticipated that the target binding can be detected by extending the surface-bound probe by one or more nucleotides using a DNA polymerase in which the nucleotide triphosphates (dNTPs) possess a detectable moiety such as a fluorescent dye (see J. M. Shumaker et al., Hum. Mutation (1996) 7:346-354).

Also, references cited are incorporated herein by reference as if each reference were individually incorporated herein by reference. The teachings of the references are therefore incorporated in their entirety.

We claim:

1. A method of detecting a binding event between a probe and a target sample comprising steps of:
contacting unstructured nucleic acid (UNA) targets derived from at least a first and second sample with a first and second UNA probe;
wherein the UNA targets corresponding to the same gene of interest derived from the first and second samples comprise different UNA nucleotide compositions;
wherein the nucleotide composition of the first UNA probe is selected to specifically hybridize to the UNA target derived from the first sample and not to the UNA target derived from the second sample, both UNA targets corresponding to the same gene of interest; and
the nucleotide composition of the second UNA probe is selected to specifically hybridize to the UNA target derived from the second sample and not to the UNA target derived from the first sample, both UNA targets corresponding to the same gene of interest;
detecting the extent of hybridization between the UNA targets derived from the first sample and the first UNA probe, and between the UNA targets derived from the second sample and the second UNA probe.

2. The method of claim 1, wherein the hybridization specificity of the UNA targets derived from the first and second samples for their respective first and second UNA probes is due to mutually exclusive base-pairing properties of the different nucleotide compositions of the UNA targets and UNA probes.

3. The method of claim 2, wherein the UNA target derived from the first sample comprises the nucleotides; D, G, C, 2-thioT and the first UNA probe comprises the nucleotides; A, G, C, T and the UNA target derived from the second sample comprises the nucleotides; A, G, 2-thioC, T and the second UNA probe comprises the nucleotides; D, I, C, T.

4. The method of claim 2, wherein the UNA target derived from the first sample comprises the nucleotides; A, G, C, 2-thioT and the first UNA probe comprises the nucleotides; A, G, C, T and the UNA target derived from the second sample comprises the nucleotides; A, G, 2-thioC, T and the second UNA probe comprises the nucleotides; D, I, C, T.

5. The method of claim 2, wherein the UNA target derived from the first sample comprises the nucleotides; A, G, C, 2-thioT and the first UNA probe comprises the nucleotides; A, I, C, T and the UNA target derived from the second sample comprises the nucleotides; A, G, P, T and the second UNA probe comprises the nucleotides; D, G, C, T.

6. The method of claim 2, wherein the UNA target derived from the first sample comprises the nucleotides; A, G, C, T and the first UNA probe comprises the nucleotides; A, G, C, 2-thioT and the UNA target derived from the second sample comprises the nucleotides; D, I, C, T and the second UNA probe comprises the nucleotides; A, G, 2-ThioC, T.

7. The method of claim 2, wherein the UNA target derived from the first sample comprises the nucleotides; A, I, C, T and the first UNA probe comprises the nucleotides; A, G, C, 2-thioT and the UNA target derived from the second sample comprises the nucleotides; D, G, C, T and the second UNA probe comprises the nucleotides; A, G, P, T.

8. The method of claim 2, wherein the UNA target derived from the first sample comprises the nucleotides; D, G, C, 2-thioT and the first UNA probe comprises the nucleotides; A, G, C, T and the UNA target derived from the second sample comprises the nucleotides; A, G, 2-thioC, T and the second UNA probe comprises the nucleotides; D, I, C, T.

9. The method of claim 1, wherein a plurality of pairs of probes comprising the first and second UNA probes are provided for a plurality of genes of interest.

10. The method of claim 9, wherein the plurality of pairs of probes is stably associated with the surface of a solid support.

11. The method of claim 1, wherein the step of contacting comprises simultaneously contacting the first target UNA sample, the second target UNA sample, the first UNA probe, and the second UNA probe.

12. The method claim 1, wherein the step of contacting occurs in a single vessel.

13. The method of claim 1, wherein the first and second target UNA samples comprise a label and the step of detecting comprises detecting the presence of the label.

14. The method of claim 13, wherein the label is selected from the group consisting of isotopic, fluorescent, electrochemical, redox, calorimetric, bio-conjugate and enzymatic labels.

15. The method of claim 1, wherein at least one probe is labeled.

16. The method of claim 15, wherein the label is selected from the group consisting of isotopic, fluorescent, electrochemical, redox, calorimetric, bio-conjugate and enzymatic labels.

17. The method of claim 15, wherein target UNA binding changes a property of the probe label and the step of detecting comprises detecting said change.

18. The method of claim 17, wherein a change in the property of the label comprises a change in an electrical property of the label.

19. The method of claim 17, wherein a change in the property of the label comprises a change in an optical property of the label.

20. The method of claim 1, wherein the extent of hybridization is determined by incorporation of a label subsequent to hybridization, and the step of detecting comprises detecting the presence of the label.

21. The method of claim 19, wherein the label is a double-stranded specific nucleic acid intercalating dye.

22. The method of claim 19, wherein the label is incorporated during a polymerase extension reaction.

23. The method of claim 1, wherein the plurality of UNA probes are derived from messenger RNA.

24. The method of claim 9, wherein the plurality of UNA probes is derived from natural DNA.

25. The method of claim 9, wherein the plurality of UNA probes are synthesized by a chemical method.

26. The method of claim 9, wherein the plurality of UNA probes are synthesized by an enzymatic method.

27. The method of claim 1, wherein the target UNAs in the first and second samples are derived from messenger RNA.

28. The method of claim 1, wherein the target UNAs in the first and second samples are derived from DNA.

29. The method of claim 10, wherein the method further comprises the step of washing the solid support of unbound UNA targets prior to the step of detecting.

30. The method of claim 1, wherein the extent of hybridization between the UNA targets derived from the first and second samples and their respective first and second UNA probes is a measure of the differential expression of a gene of interest within the first and second samples.

31. A method of producing an array of UNA probes stably associated with a surface of a solid support, wherein the UNA probes are designed in pairs comprising at least a first and a second probe, wherein the first and second probes have different UNA nucleotides and are capable of hybridizing to UNA targets from different samples, the method comprising the steps of:
selecting at least one gene that is likely to be present in different samples; and
generating at least a pair of UNA probes for the gene, wherein the first probe of the pair is capable of hybridizing to the target derived from the first sample and not the target UNA derived from the second sample, and the second probe of the pair is capable of hybridizing to the target UNA derived from the second sample and not the target UNA derived from first sample; and
creating an array containing at least the pair of UNA probes.

32. The method of claim 31, wherein the UNA probes are derived from messenger RNA.

33. The method of claim 31, wherein the UNA probes are derived from natural DNA.

34. The method of claim 31, wherein the pairs of UNA probes are synthesized by a chemical method.

35. The method of claim 31, wherein the pairs of UNA probes are synthesized by an enzymatic method.

36. The method of claim 31, wherein at least one of the first and second probes is labeled.

37. The method of claim 36, wherein the label is selected from the group consisting of isotopic, fluorescent, electrochemical, redox, calorimetric, bio-conjugate and enzymatic labels.

38. The method of claim 31, wherein in the step of selecting the at least one gene is a plurality of genes.

39. The method of claim 31 wherein in the step of generating, the at least one pair is a plurality of pairs.

40. An array of a plurality of UNA probes, wherein the UNA probes are designed in pairs, wherein each member of a pair has a different UNA nucleotide content and is capable of detecting a UNA target corresponding to the same gene derived from different samples, wherein the plurality of UNA probes are:
stably associated with a surface of a solid support; and
synthesized such that the first member of the pair of UNA probes can be differentiated from the second member of the pair by a difference in UNA nucleotide content, wherein the first member of the pair of UNA probes is capable of hybridizing to a UNA target derived from the first sample and cannot hybridize to a UNA target derived from second sample, and the second member of the pair of UNA probes is capable of hybridizing to a UNA target derived from the second sample and cannot hybridize to the UNA target derived from the first sample.

41. The array of claim 40, wherein at least the first member of the probe is labeled.

42. The method of claim 41, wherein the label is selected from the group consisting of isotopic, fluorescent, electro chemical, redox, colorimetric, bio-conjugate and enzymatic labels.

43. The array of claim 42, wherein the plurality of UNA probes are derived from messenger RNA.

44. The array of claim 42, wherein the plurality of UNA probes are derived from natural DNA.

45. The array of claim 42, wherein the plurality of UNA probes are deposited on the surface of the solid support before a stable association is formed.

46. The array of claim 42, wherein the plurality of UNA probes are synthesized, in situ on the solid support.

47. A kit for carrying out differential gene expression analysis comprising:
an array of a plurality of UNA probes, wherein the UNA probes are designed in pairs, wherein each member of a pair has a different UNA nucleotide content and is capable of detecting a UNA target corresponding to the same gene derived from different samples, wherein the plurality of UNA probes are:
stably associated with a surface of a solid support; and
synthesized such that the first member of the pair of UNA probes can be differentiated from the second member of the pair by a difference in UNA nucleotide content, wherein the first member of the pair of UNA probes is capable of hybridizing to a UNA target derived from the first sample and cannot hybridize to a UNA target derived from second sample, and the second member of the pair of UNA probes is capable of hybridizing to a UNA target derived from the second sample and cannot hybridize to the UNA target derived from the first sample.

48. The kit of claim 47, further comprising a vessel for containing the array.

49. The kit of claim 47, wherein the array is incorporated into a multiwell configuration.

50. The kit of claim 47, wherein the array is incorporated into a biochip configuration.

51. The kit of claim 47, further comprising target sample generation reagents.

52. The kit of claim 47, further comprising reagents used in the binding step.

53. The kit of claim 47, further comprising signal producing system members.

54. The kit of claim 47, wherein at least one probe is labeled.

55. The method of claim 54, wherein the label is selected from the group consisting of isotopic, fluorescent, electrochemical, redox, calorimetric, bio-conjugate and enzymatic labels.

56. A system for detecting a binding event on a surface comprising at least one probe attached to the surface; and
one or more UNA targets derived from two or more samples, wherein the targets corresponding to the same gene comprise different UNA nucleotide compositions, wherein
the UNA targets derived from the two or more samples comprise different UNA nucleotide compositions and can compete for hybridization to the same surface probe; and
two or more labeling probes having a different UNA chemistry, wherein one UNA labeling probe is capable of hybridizing to a UNA target derived from one sample and cannot hybridize to a UNA target derived from the second sample, and the second UNA labeling probe is capable of hybridizing to a UNA target derived from the second sample and cannot hybridize to the UNA target derived from the first sample; and
wherein each labeling probe is attached to a detectable moiety.

57. The system of claim 56, wherein the surface is a microarray.

58. The system of claim 56, wherein the two or more labeling probes having a different UNA chemistry correspond to the same region of complementarity.

59. The system of claim 56, wherein the two or more labeling probes having a different UNA chemistry correspond to a different region of complementarity.

60. The system of claim 56, wherein the at least one probe attached to a surface, the one or more UNA targets derived from two or more samples, and the two or more labeling probes are simultaneously present before hybridization has occurred.

61. A method for detecting a binding event on a microarray comprising the steps of:
providing at least one array probe on an array;
contacting the array probe with targets derived from at least two samples, wherein the targets are UNAs that competitively hybridize to the same array probe;
contacting the target with labeling probes, wherein each labeling probe has a different UNA chemistry that directs sample -specific hybridization to the target, wherein each labeling probe is attached to a different detectable moity; and
detecting the labeling probes hybridized to the targets that are hybridized to the array probe.

62. The method of claim 61, further comprising the step of detecting the ratios of targets in the sample that are hybridized to the array probe by quantifying the ratios of the different detectable moieties on the labeling probes that are hybridized to said targets.
